(19) United States
(12) Patent Application Publication
(10) Pub. No.: US 2010/0305116 A1
(43) Pub. Date: Dec. 2, 2010

(54) Compounds Comprising a Cyclobutoxy Group

(75) Inventors: Sylvain Celanire, Reignier (FR); Frederic Denonne, Brussels (BE); Anne Valade, Brussels (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: UCB PHARMA, S.A., Brussels (BE)

(21) Appl. No.: 12/596,545

(22) PCT Filed: Apr. 14, 2008
(86) PCT No.: PCT/EP08/54496

§ 371 Date: Aug. 18, 2010

Related U.S. Application Data
(60) Provisional application No. 60/913,351, filed on Apr. 23, 2007.

(30) Foreign Application Priority Data
Apr. 19, 2007 (EP) 07007968.6

(51) Int. Cl. A61K 31/5377; C07D 401/14; A61K 31/4545; C07D 207/22; A61K 31/40; C07D 413/14; C07D 413/12; A61K 31/454; C07D 211/22; C07D 265/30; C07D 241/04; A61P 25/00; A61P 25/28; A61P 9/00; A61P 29/00

(52) U.S. Cl. 514/230.8; 546/187; 514/316; 548/557; 514/426; 544/129; 514/236.8; 546/209; 514/326; 546/236; 544/173; 544/398

(57) ABSTRACT

The present invention relates to compounds comprising a cyclobutoxy group, processes for preparing them, phar-maceutical compositions comprising said compounds and their use as pharmaceuticals.

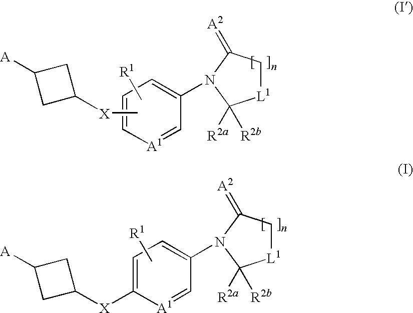

The present invention relates to compounds comprising a cyclobutoxy group, processes for preparing them, pharmaceutical compositions comprising said compounds and their use as pharmaceuticals.

The histamine H₃ receptor has been known for several years and identified pharmacologically in 1983 by Arrang, J. M. et al. (Nature 1983, 302, 832-837). Since the cloning of the human histamine H₃ receptor in 1999, histamine H₃ receptors have been successively cloned by sequence homology from a variety of species, including rat, guinea pig, mouse and monkey.

Histamine H₃-receptor agonists, antagonists and inverse agonists have shown potential therapeutic applications as described in the literature, for example by Stark, H. in Exp. Opin. Ther. Patents 2003, 13, 851-865, and by Leurs R. et al. in Nature Review Drug Discovery 2005, 4, 107-120.

The histamine H₃ receptor is predominantly expressed in the mammalian central nervous system but can also be found in the autonomic nervous system. Evidence has been shown that the histamine H₃ receptor displays high constitutive activity, which activity occurs in the absence of endogenous histamine or of a H₃-receptor agonist. Thus, a histamine H₃-receptor antagonist and/or inverse agonist could inhibit this activity.

The general pharmacology of histamine H₃ receptor, including H₃-receptor subtypes, has been reviewed by Hancock, A. A in Life Sci. 2003, 73, 3043-3072. The histamine H₃ receptor is not only considered as a presynaptic autoreceptor on histaminergic neurons, but also as a heteroreceptor on non-histaminergic neurons (Barnes, W. et al., Eur. J. Pharmacol. 2001, 431, 215-221). Indeed, the histamine H₃ receptor has been shown to regulate the release of histamine but also of other important neurotransmitters, including acetylcholine, dopamine, serotonin, norepinephrin and γ-aminobutyric acid (GABA).

Thus, the histamine H₃ receptor is of current interest for the development of new therapeutics and the literature suggests that novel histamine H₃-receptor antagonists or inverse agonists may be useful for the treatment and prevention of diseases or pathological conditions of the central nervous system including Mild Cognitive Impairment (MCI), Alzheimer's disease, learning and memory disorders, cognitive disorders, attention deficit disorder (ADD), attention-deficit hyperactivity disorder (ADHD), Parkinson's disease, schizophrenia, dementia, depression, epilepsy, seizures or convulsions, sleep/wake disorders, narcolepsy, pain and/or obesity.

H₃-receptor ligands alone or in combination with an acetylcholinesterase inhibitor may also be useful in the treatment of cholinergic-deficit disorders, Mild Cognitive Impairment and Alzheimer's disease as reported by Morisset, S. et al. in Eur. J. Pharmacol. 1996, 315, R1-R2.

H₃-receptor ligands, alone or in combination with a histamine H₁-receptor antagonist may be useful for the treatment of upper airway allergic disorders, as reported by McLeod, R. et al. in J. Pharmacol. Exp. Ther. 2003, 305, 1037-1044.

H₃-receptor ligands, alone or in combination with a serotonine reuptake inhibitor may be useful for the treatment of depression, as reported by Keith, J. M. et al in Bioorg. Med. Chem. Lett. 2007, 17, 702-706.

As described in international patent application WO 02/072093, H₃-receptor ligands alone or in combination with a muscarinic receptor ligand and particularly with a muscarinic M₂-receptor antagonist, may be useful for the treatment of cognitive disorders, Alzheimer's disease, attention-deficit hyperactivity disorder.

H₃-receptor ligands may also be useful in the treatment of sleep/wake and arousal/vigilance disorders such as hypersomnia, and narcolepsy according to Passani, M. B. et al. in Trends Pharmacol. Sci. 2004, 25(12), 618-625.

In general, H₃-receptor ligands, and particularly H₃-receptor antagonists or inverse agonists may be useful in the treatment of all types of cognitive-related disorders as reviewed by Hancock, A. A and Fox, G. B. in Expert Opin. Invest. Drugs 2004, 13, 1237-1248.

In particular, histamine H₃-receptor antagonists or inverse agonists may be useful in the treatment of cognitive dysfunctions in diseases such as Mild Cognitive Impairment, dementia, Alzheimer's disease, Parkinson's disease, Down's syndrome as well as in the treatment of attention-deficit hyperactivity disorder (ADHD) as non-psychostimulant agents (see for example Witkin, J. M. et al., Pharmacol. Ther. 2004, 103(1), 1-20).

H₃-receptor antagonists or inverse agonists may also be useful in the treatment of psychotic disorders such as schizophrenia, migraine, eating disorders such as obesity, inflammation, pain, anxiety, stress, depression and cardiovascular disorders, in particular acute myocardial infarction.

There is therefore a need to manufacture new compounds which can potentially act as H₃-receptor ligands.

Early literature reports (e.g. Ali, S. M. et al., J. Med. Chem. 1999, 42, 903-909 and Stark, H. et al., Drugs Fut. 1996, 21, 507-520) describe that an imidazole function is essential for high affinity histamine H₃-receptor ligands; this is confirmed, for example, by United States patents U.S. Pat. No. 6,506,756B2, U.S. Pat. No. 6,518,287B2, U.S. Pat. No. 6,528,522B2 and U.S. Pat. No. 6,762,186B2 which relate to substituted imidazole compounds that have H₃-receptor antagonist or dual histamine H₁-receptor and H₃-receptor antagonist activity.

International patent application WO 02/12214 relates to non-imidazole aryloxyalkylamines for the treatment of disorders and conditions mediated by the histamine receptor.

International patent application WO 02/074758 relates to bicyclic heterocyclic derivatives comprising an amine moiety and reported as H₃-receptor ligands.

International patent application WO 2004/056369 relates to benzodiazepine derivatives for the treatment of neurological disorders.

International patent application WO 2005/007644 relates to heteroaryloxy nitrogenous saturated heterocyclic derivatives which exhibit histamine receptor H₃ antagonist or inverse agonist activity.

International patent application WO 03/089409 describes compounds comprising a lactam moiety and having affinity at 5HT_{2C} receptor.

Compounds comprising a lactam moiety are described as synthesis intermediates by J. L. Neumeyer et al. in J. Med. Chem. 1967, 10, 615-620.

Selvakumar N. et al. in Bioorg. Med. Chem. Lett. 2003, 13, 4169-4172 describe oxazolidinone derivatives as antibacterial agents.

International patent application WO 2006/136924 describes a class of phenoxycyclobutyl derivatives as H₃-receptor antagonists. US patent application US 2005/171181 discloses cyclobutyl-arylamines as H₃-receptor modulators.

International patent application WO 2006/132914 and US patent application US 2007/0066588 describe cyclobutyl amine derivatives as H₃-receptor modulators.

It has now surprisingly been found that compounds of formula (I) may act as H₃-receptor ligands and therefore may demonstrate therapeutic properties for one or more pathologies mentioned below.

The present invention relates to compounds of formulae (I′) and (I), geometrical isomers, enantiomers, diastereoisomers, pharmaceutically acceptable salts and all possible mixtures thereof,

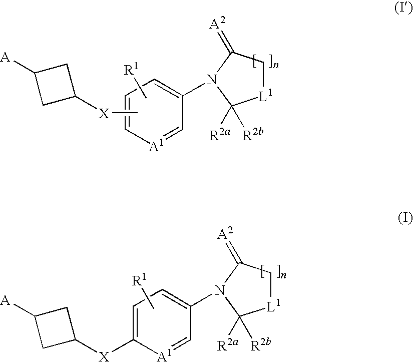

wherein

### A¹ is CH, C-halogen or N;

A² is oxygen or sulfur;
X is O, S, NH or N(C₁₋₄ alkyl);
R¹ is hydrogen, halogen, C₁₋₄ alkyl or C₁₋₄ alkoxy;
R^{2a} is hydrogen, substituted or unsubstituted C₁₋₆ alkyl, substituted or unsubstituted C₁₋₆-alkyl cycloalkyl, substituted or unsubstituted aryl, substituted or unsubstituted C₂₋₆ alkenyl, substituted or unsubstituted heteroaryl, substituted or unsubstituted C₃₋₈ cycloalkyl, substituted or unsubstituted 3-8-membered heterocycloalkyl, substituted or unsubstituted acyl, substituted or unsubstituted C₁₋₆-alkyl aryl, substituted or unsubstituted C₁₋₆-alkyl heteroaryl, substituted or unsubstituted C₁₋₆-alkyl heterocycloalkyl, substituted or unsubstituted alkoxycarbonyl, substituted or unsubstituted aminocarbonyl, substituted or unsubstituted C₁₋₆-alkyl acyl, substituted or unsubstituted C₁₋₆-alkyl alkoxy, substituted or unsubstituted C₁₋₆-alkyl alkoxycarbonyl, substituted or unsubstituted C₁₋₆-alkyl aminocarbonyl, substituted or unsubstituted C₁₋₆-alkyl acylamino, substituted or unsubstituted C₁₋₆-alkyl ureido, substituted or unsubstituted C₁₋₆-alkyl aminocarbonyloxy, substituted or unsubstituted C₁₋₆-alkyl aminocarbonylthio, substituted or unsubstituted C₁₋₆-alkyl carbamate, substituted or unsubstituted C₁₋₆-alkyl amino, substituted or unsubstituted C₃₋₈-cycloalkyl amino, substituted or unsubstituted C₁₋₆-alkyl hydroxy or cyano;
R^{2b} is hydrogen, C₁₋₈ alkyl or C₃₋₈ cycloalkyl;
or R^{2a} and R^{2b} are linked together to form a substituted or unsubstituted C₃₋₈ cycloalkyl
or a substituted or unsubstituted 3-8 membered heterocycloalkyl;
A is a substituted or unsubstituted aliphatic or cyclic amino group which is linked to the cyclobutyl group via an amino nitrogen;
L¹ is —(O)ᵥ—(CR^{9a}R^{9b})ₘ—(CH₂)_{z};
R^{9a} is hydrogen or C₁₋₈ alkyl;
R^{9b} is a C₁₋₆-alkyl aryl or C₁₋₈ alkyl;
n is an integer equal to 0 or 1;
v is an integer equal to 0 or 1;
m is an integer equal to 0 or 1;
z is an integer equal to 0, 1, 2 or 3.

The term “alkyl”, as used herein, is a group which represents saturated, monovalent hydrocarbon radicals having straight (unbranched) or branched moieties, or combinations thereof, and containing 1-8 carbon atoms, preferably 1-6 carbon atoms; more preferably alkyl groups have 1-4 carbon atoms.

Usually, according to the present invention, alkyl groups are not substituted. Preferred such alkyl groups according to the present invention are methyl, ethyl, n-propyl and isopropyl.

Some alkyl groups may be substituted by 1 to 5 halogen atoms. Examples of such an alkyl groups are trifluoromethyl and trifluoroethyl.

The term “halogen”, as used herein, represents an atom of fluorine, chlorine, bromine, or iodine. Preferred halogens are chlorine and fluorine.

The term “hydroxy”, as used herein, represents a group of formula —OH.

The term “C₁₋₆-alkyl hydroxy”, as used herein, refers to an alkyl as defined above substituted by a hydroxy. Suitable “C₁₋₆-alkyl hydroxy” groups include hydroxymethyl and 2-hydroxyethyl.

The term “C₃₋₈ cycloalkyl”, as used herein, represents a monovalent group of 3 to 8 carbon atoms derived from a saturated cyclic hydrocarbon. Typical C₃₋₈ cycloalkyl groups according to the present invention are cyclopropyl, cyclobutyl, cyclopentyl and cyclohexyl. Likewise the term “C₃₋₁₄ cycloalkyl” refers to a monovalent group of 3 to 14 carbon atoms derived from a saturated cyclic hydrocarbon.

The term “C₁₋₆-alkyl cycloalkyl”, as used herein, refers to a C₁₋₆ alkyl having a cycloalkyl substitutent as defined here above. Examples of “C₁₋₆-alkyl cycloalkyl” according to the invention are cyclopropylmethyl, cyclopentylmethyl and cyclohexylmethyl.

The term “alkylene”, as used herein, represents a group of formula —(CH₂)ₓ— in which x is comprised between 2 and 6, preferably comprised between 3 and 6.

The term “methylene” as used herein represents a group of formula —CH₂—.

The term “C₂₋₆ alkenyl” refers to alkenyl groups preferably having from 2 to 6 carbon atoms and having at least 1 or 2 sites of alkenyl unsaturation. Preferred alkenyl groups include ethenyl (vinyl, —CH═CH₂), n-2-propenyl (allyl, —CH₂—CH═CH₂) and the like.

The term “C₂₋₆ alkynyl” refers to alkynyl groups preferably having from 2 to 6 carbon atoms and having at least 1 to 2 sites of alkynyl unsaturation. Preferred alkynyl groups include ethynyl (—C≡CH), propargyl (—CH₂—C≡CH), and the like.

The term “aryl” as used herein, refers to an unsaturated aromatic carbocyclic group of from 6 to 14 carbon atoms having a single ring (e.g., phenyl) or multiple condensed rings (e.g., naphthyl). The “aryl” groups may be unsubstituted or substituted by 1 to 4 substituents independently selected from halogen, C₁₋₄ alkyl or C₁₋₄ alkoxy as defined herein. Suitable aryl groups include phenyl, 2-fluorophenyl, 3-fluorophenyl, 4-fluorophenyl, 2,4-difluorophenyl, 3,4-difluorophenyl, 3,5-difluorophenyl, 3-methoxyphenyl, 4-(trifluoromethyl)phenyl, 4-methylphenyl, 1,3-benzodioxol-5-yl, and 4-chlorophenyl.

The term “C₁₋₆-alkyl aryl”, as used herein, refers to a group of formula —R^{e}-aryl in which R^{e} is a C₁₋₆ alkyl. Examples of “C₁₋₆-alkyl aryl” according to the present invention are benzyl, 4-fluorobenzyl and 4-chlorobenzyl.

The term “heteroaryl” as used herein represents an aryl group as defined here above wherein one or more of the carbon atoms have been replaced by a heteroatom as defined herein. Examples of heteroaromatic groups are pyridyl, pyrrolyl, furyl, thienyl, imidazolyl, triazolyl and the like.

The term “C₁₋₆-alkyl heteroaryl” refers to a C₁₋₆ alkyl having a heteroaryl substituent as defined hereabove. Examples include 2-furylmethyl, (2-methyl-1H-imidazol-1-yl)methyl and (1H-1,2,4-triazol-1-yl)methyl.

The term “alkoxy”, as used herein, represents a group of formula —OR^{a} wherein R^{a} is an alkyl or an aryl group, as defined above. Usually, according to the present invention, alkyl group of alkoxy group is not substituted. Examples of alkoxy groups are methoxy, 4-fluorophenoxy and 3,4-difluorophenoxy.

The term “C₁₋₆-alkyl alkoxy”, as used herein, refers to a C₁₋₆ alkyl group having an alkoxy substituent as defined hereabove. Examples of “C₁₋₆-alkyl alkoxy” are (4-fluorophenoxy)methyl and (3,4-difluorophenoxy)methyl.

The term “carbonyl”, as used herein represents a group of formula —(C═O)—.

The term “acyl”, as used herein, represents a group of formula —C(═O)R^{b} wherein R^{b} is an alkyl, a C₃₋₈ cycloalkyl or an aryl group, as defined here above. Preferred acyl group is acetyl or cyclopropylcarbonyl. The term “arylcarbonyl” as used herein, represents an acyl group as defined here above wherein R^{b} is an aryl group as defined here above.

The term “C₁₋₆-alkyl acyl” as used herein refers to a C₁₋₆ alkyl having an acyl substituent as defined here above, including 3-oxobutyl and the like.

The term “heterocycloalkyl” as used herein represents a cycloalkyl as defined here above wherein one, two or three carbon atoms are replaced by one, two or three O, S or N. Particularly, the heterocycloalkyl is a 3 to 14 membered, preferably 3 to 8 membered heterocycloalkyl, i.e. a heterocycloalkyl wherein the cycloalkyl is a C₃₋₁₄ cycloalkyl, preferably C₃₋₈ cycloalkyl. The heterocycloalkyl may be unsubstituted or substituted by any suitable group including, but not limited to, one or more, typically one, two or three, moieties selected from alkyl, amino, cycloalkyl, hydroxy, alkoxy, acyl, aryl and halogen. Examples of heterocycloalkyl are piperidinyl, 4,4-difluoropiperidinyl, morpholinyl, pyrrolidinyl, 3,3-difluoropyrrolidinyl, 3-(dimethylamino)pyrrolidinyl and 4-cyclobutylpiperazinyl as well as azepanyl, 4-(cyclohexylmethyl)-piperazinyl, 4-(cyclopentyl)piperazinyl, 4-(isopropyl)-piperazinyl, 2,6-dimethylpiperidinyl, 2-methylpiperidinyl, (2S)-2-methylpyrrolidinyl, (2R)-2-methylpyrrolidinyl, 4-methylpiperidinyl, 2-methylpyrrolidinyl, 1-benzylpyrrolidinyl, 4-benzylpiperidinyl, 3-phenylpiperidinyl, (2-hydroxymethyl)pyrrolidinyl, (4aR,8aS)-octahydroisoquinolinyl, octahydroisoquinolinyl, 2,6-dimethylmorpholinyl, cis-2,6-dimethylmorpholinyl, thiomorpholinyl, 1,1-dioxidothiomorpholinyl, 1-acetylpyrrolidinyl, (2S)-2-(pyrrolidin-1-ylmethyl)pyrrolidinyl, azaspiro[4,4]nonyl, azaspiro[5,5]undecyl, azocanyl and 3,5-dimethylpiperidinyl.

The term “C₁₋₆-alkyl heterocycloalkyl”, as used herein, refers to a C₁₋₆ alkyl substituted by a heterocycloalkyl as defined here above. Examples of “C₁₋₆-alkyl heterocycloalkyl” according to the present invention are piperidin-1-ylmethyl, (4,4-difluoropiperidin-1-yl)methyl, morpholin-4-ylmethyl, pyrrolidin-1-ylmethyl and (3,3-difluoropyrrolidin-1-yl)methyl as well as azepan-1-ylmethyl, [4-(cyclohexylmethyl)piperazin-1-yl]methyl, [4-(cyclopentyl)piperazin-1-yl]methyl, 2-[4-(cyclopentyl)piperazin-1-yl]ethyl, (4-(isopropyl)piperazin-1-yl)methyl, 2-piperidin-1-ylethyl, (2,6-dimethylpiperidin-1-yl)methyl, (2-methylpiperidin-1-yl)methyl, (4-methylpiperidin-1-yl)methyl, (2-methylpyrrolidin-1-yl)methyl, (4aR,8aS)-octahydroisoquinolin-2(1H)-ylmethyl, (2,6-dimethylmorpholin-4-yl)methyl, [cis-2,6-dimethylmorpholin-4-yl]methyl, thiomorpholin-4-ylmethyl and (1,1-dioxidothiomorpholin-4-yl)methyl.

The term “heterocycloalkyl acyl” refers to a heterocycloalkyl group having an acyl substituent as defined here above.

The term “amino”, as used herein, represents an aliphatic group of formula —NR^{c}R^{d} wherein R^{c} and R^{d} are independently hydrogen, “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl”, “C₁₋₆-alkyl heteroaryl”, “C₁₋₆-alkyl cycloalkyl” or “C₁₋₆-alkyl heterocycloalkyl” groups; or a or cyclic group of formula —NR^{c}R^{d} wherein R^{c} and R^{d} are linked together with N to form a 3 to 14 membered, preferably 3 to 8 membered heterocycloalkyl, as defined hereinabove.

Examples of “amino” groups are piperidin-1-yl, 4,4-difluoropiperidin-1-yl, morpholin-4-yl, pyrrolidin-1-yl, 3,3-difluoropyrrolidiny-1-yl, (2,2,2-trifluoroethyl)amino, (2,2,2-trifluoroethyl)(methyl)amino, dimethylamino, diethylamino, cyclobutylamino, (4-fluorophenyl)amino, (4-fluorophenyl)(methyl)amino as well as cyclohexylmethylamino, (cyclohexylmethyl)(cyclopropylmethyl)amino, (cyclopropylmethyl)(propyl)amino, cyclo-hexylamino, cyclopentylamino, anilino, (4-fluorobenzyl)amino, (cyclohexylmethyl)(cyclo-propylcarbonyl)-amino, (2-fluorophenyl)amino, (3-fluorophenyl)amino, (2,4-difluoro-phenyl)amino, (3-methoxyphenyl)amino, [4-(trifluoromethyl)phenyl]amino, (4-methyl-phenyl)amino, (3,4-difluorophenyl)amino, (3,5-difluorophenyl)amino, 1,3-benzodioxol-5-ylamino, (4-fluorophenyl)(methyl)amino, dibenzylamino, amino, acetylamino, azepan-1-yl, 4-(cyclohexylmethyl)piperazin-1-yl, 4-(cyclopentyl)piperazin-1-yl, 4-(isopropyl)piperazin-1-yl, 2,6-dimethylpiperidin-1-yl, 2-methylpiperidin-1-yl, (2S)-2-methylpyrrolidin-1-yl, (2R)-2-methylpyrrolidin-1-yl, 4-methylpiperidin-1-yl, 2-methylpyrrolidin-1-yl, 4-benzylpiperidin-1-yl, 3-phenylpiperidin-1-yl, (2-hydroxymethyl)pyrrolidin-1-yl, (4aR,8aS)-octahydroisoquinolin-2(1H)-yl, octahydroisoquinolin-2(1H)-yl, 2,6-dimethylmorpholin-4-yl, cis-2,6-dimethylmorpholin-4-yl, thiomorpholin-4-yl, 1,1-dioxidothiomorpholin-4-yl, (2S)-2-(pyrrolidin-1-ylmethyl)pyrrolidin-1-yl, 1-azaspiro[4,4]non-1-yl, 2-azaspiro[5,5]undec-2-yl, azocan-1-yl and 3,5-dimethylpiperidin-1-yl.

The term “C₁₋₆-alkyl amino”, as used herein, represents a C₁₋₆ alkyl group substituted by an amino group as defined above. Examples of “C₁₋₆-alkyl amino” according to the present invention are piperidin-1-ylmethyl, (4,4-difluoropiperidin-1-yl)methyl, morpholin-4-ylmethyl, pyrrolidin-1-ylmethyl, (3,3-difluoropyrrolidiny-1-yl)methyl, [(2,2,2-trifluoroethyl)amino]methyl as well as [(cyclohexylmethyl)amino]methyl, [(cyclohexyl-methyl)(cyclopropylmethyl)amino]methyl, [(cyclopropylmethyl)(propyl)amino]methyl, (cyclobutylamino)methyl, (cyclohexylmethylamino)methyl, (cyclopentyl-amino)methyl, (diethylamino)methyl, anilinomethyl, [(4-fluorobenzyl)amino]methyl, [(4-fluorophenyl)-amino]methyl, [(cyclohexylmethyl)(cyclopropylcarbonyl)amino]methyl, [(2-fluorophenyl)-amino]-methyl, [(3-fluorophenyl)amino]methyl, [(2,4-difluorophenyl)-amino]methyl, [(3-methoxyphenyl)amino]methyl, {[4-(trifluoromethyl)phenyl]amino}methyl, [(4-methyl-phenyl)amino]methyl, [(3,4-difluorophenyl)amino]methyl, [(3,5-difluorophenyl)-amino]methyl, [(2,2,2-trifluoroethyl)amino]methyl, [1,3-benzodioxol-5-ylamino]methyl, [(4-fluorophenyl)-(methyl)amino]methyl, (dibenzylamino)methyl, aminomethyl, (acetylamino)-methyl, azepan-1-ylmethyl, [4-(cyclohexylmethyl)piperazin-1-yl]methyl, [4-(cyclopentyl)-piperazin-1-yl]methyl, 2-[4-(cyclopentyl)piperazin-1-yl]ethyl, (4-(isopropyl)piperazin-1-yl)methyl, 2-piperidin-1-ylethyl, (2,6-dimethylpiperidin-1-yl)methyl, (2-methylpiperidin-1-yl)methyl, (4-methyl-piperidin-1-yl)methyl, (2-methylpyrrolidin-1-yl)methyl, (4aR,8aS)-octahydroisoquinolin-2(1H)-ylmethyl, (2,6-dimethylmorpholin-4-yl)methyl, [cis-2,6-dimethylmorpholin-4-yl]methyl, thiomorpholin-4-ylmethyl and (1,1-dioxidothiomorpholin-4-yl)methyl.

The term “aminocarbonyl” as used herein refers to a group of formula —C(O)NR^{c}R^{d} wherein R^{c} and R^{d} are as defined here above for the amino group. Examples of “aminocarbonyl” include (diethylamino)carbonyl, (cyclobutylamino)carbonyl, piperidin-1-ylcarbonyl, (4,4-difluoropiperidin-1-yl)carbonyl, [(2,2,2-trifluoroethyl)amino]carbonyl, [methyl(2,2,2-trifluoroethyl)amino]carbonyl, [(4-fluorophenyl)amino]carbonyl, [(4-fluorophenyl)(methyl)amino]carbonyl, morpholin-4-ylcarbonyl and (3,3-difluoropyrrolidin-1-yl)carbonyl.

The term “C₁₋₆-alkyl aminocarbonyl” as used herein, refers to a C₁₋₆ alkyl substituted by an aminocarbonyl as defined hereabove.

The term “C₃₋₈-cycloalkyl amino”, as used herein, represents a C₃₋₈ cycloalkyl group substituted by an amino group as defined above.

The term “acylamino”, as used herein refers to a group of formula —NR^{c}C(O)R^{d} wherein R^{c} and R^{d} are as defined hereabove for the amino group.

The term “C₁₋₆-alkyl acylamino”, as used herein refers to a C₁₋₆ alkyl substituted by an acylamino as defined hereabove.

The term “carboxy”, as used herein represents a group of formula —COOH.

The term “C₁₋₆-alkyl carboxy”, as used herein refers to a C₁₋₆ alkyl substituted by a carboxy group including 2-carboxyethyl and the like.

The term “cyano”, as used herein represents a group of formula —CN.

The term “alkoxycarbonyl” refers to the group —C(O)OR^{g} wherein R^{g} includes “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkyl cycloalkyl”, “C₁₋₆-alkyl heterocycloalkyl”. Examples of alkoxycarbonyl are methoxycarbonyl and ethoxycarbonyl.

The term “C₁₋₆-alkyl alkoxycarbonyl” refers to a C₁₋₆ alkyl having an alkoxycarbonyl as defined here above as substituent.

The term “ureido” as used herein refers to a group of formula —NRⁱC(O)NR^{c}R^{d} wherein Rⁱ is as defined hereabove for R^{c} or R^{d}, and R^{c} and R^{d} are as defined here above for the amino group. Rⁱ is typically hydrogen or C₁₋₄ alkyl. Examples of “ureido” include (pyrrolidin-1-ylcarbonyl)amino and methyl(pyrrolidin-1-ylcarbonyl)amino.

The term “C₁₋₆-alkyl ureido” as used herein refers to a C₁₋₆ alkyl substituted by an ureido as defined here above. Examples of “C₁₋₆-alkyl ureido” include [(pyrrolidin-1-ylcarbonyl)amino]methyl and [methyl(pyrrolidin-1-ylcarbonyl)amino]methyl.

The term “carbamate”, as used herein, refers to a group of formula —NR^{c}C(O)OR^{d} wherein R^{c} and R^{d} are as defined here above for the amino group.

The term “C₁₋₆-alkyl carbamate” as used herein refers to a C₁₋₆ alkyl substituted by a carbamate as defined here above.

The term “aminocarbonyloxy” as used herein refers to a group of formula —OC(O)N R^{c}R^{d} wherein R^{c} and R^{d} are as defined here above for the amino group. Examples of “aminocarbonyloxy” include (pyrrolidin-1-ylcarbonyl)oxy, (piperidin-1-ylcarbonyl)oxy, (morpholin-4-ylcarbonyl)oxy, [(3,3-difluoropiperidin-1-yl)carbonyl]oxy and [(4,4-difluoropiperidin-1-yl)carbonyl]oxy.

The term “C₁₋₆-alkyl aminocarbonyloxy” as used herein refers to a C₁₋₆ alkyl substituted by an aminocarbonyloxy as defined here above. Examples of “C₁₋₆-alkyl aminocarbonyloxy” include [(pyrrolidin-1-ylcarbonyl)oxy]methyl, [(piperidin-1-ylcarbonyl)oxy]methyl, [(morpholin-4-ylcarbonyl)oxy]methyl, {[(3,3-difluoropiperidin-1-yl)carbonyl]oxy}methyl and {[(4,4-difluoropiperidin-1-yl)carbonyl]oxy}methyl.

The term “aminocarbonylthio” as used herein refers to a group of formula —SC(O)N R^{c}R^{d} wherein R^{c} and R^{d} are as defined here above for the amino group.

The term “C₁₋₆-alkyl aminocarbonylthio” as used herein refers to a C₁₋₆ alkyl substituted by an aminocarbonylthio as defined here above.

The term “oxo” as used herein refers to ═O.

“Sulfonyl” refers to group “—SO₂—R” wherein R is selected from H, “aryl”, “heteroaryl”, “C₁₋₆ alkyl”, “C₁₋₆ alkyl” substituted with halogens, e.g., an —SO₂—CF₃ group, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkenyl aryl”, “C₂₋₆-alkenyl heteroaryl”, “C₂₋₆-alkynyl aryl”, “C₂₋₆-alkynylheteroaryl”, “C₁₋₆-alkyl cycloalkyl”, “C₁₋₆-alkyl heterocycloalkyl”.

“Sulfinyl” refers to a group “—S(O)—R” wherein R is selected from H, “C₁₋₆ alkyl”, “C₁₋₆ alkyl” substituted with halogens, e.g., an —SO—CF₃ group, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkenyl aryl”, “C₂₋₆-alkenyl heteroaryl”, “C₂₋₆-alkynyl aryl”, “C₂₋₆-alkynylheteroaryl”, “C₁₋₆-alkyl cycloalkyl”, “C₁₋₆-alkyl heterocycloalkyl”.

“Sulfanyl” refers to groups —S—R where R includes H, “C₁₋₆ alkyl”, “C₁₋₆ alkyl” optionally substituted with halogens, e.g a —S—CF₃ group, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkenyl aryl”, “C₂₋₆-alkenyl heteroaryl”, “C₂₋₆-alkynyl aryl”, “C₂₋₆-alkynylheteroaryl”, “C₁₋₆-alkyl cycloalkyl”, “C₁₋₆-alkyl heterocycloalkyl”. Preferred sulfanyl groups include methylsulfanyl, ethylsulfanyl, and the like.

“Substituted or unsubstituted” as used herein, unless otherwise constrained by the definition of the individual substituents, shall mean that the above set out groups, like “C₁₋₆ alkyl”, “C₂₋₆ alkenyl”, “C₂₋₆ alkynyl”, “aryl” and “heteroaryl” etc. . . . may optionally be substituted with from 1 to 5 substituents selected from the group consisting of “C₁₋₆ alkyl”, “C₂₋₆ alkenyl”, “C₂₋₆ alkynyl”, “cycloalkyl”, “heterocycloalkyl”, “C₁₋₆-alkyl aryl”, “C₁₋₆-alkyl heteroaryl”, “C₁₋₆-alkyl cycloalkyl”, “C₁₋₆-alkyl heterocycloalkyl”, “amino”, “ammonium”, “acyl”, “acyloxy”, “acylamino”, “aminocarbonyl”, “alkoxycarbonyl”, “ureido”, “carbamate”, “aryl”, “heteroaryl”, “sulfinyl”, “sulfonyl”, “alkoxy”, “sulfanyl”, “halogen”, “carboxylic acid”, trihalomethyl, cyano, hydroxy, nitro, and the like. Specific substituents are halogens (e.g fluoro or chloro) or halogenated alkyl groups like a trifluoromethyl.

In a specific embodiment, compounds of the present invention are those according to formula (I).

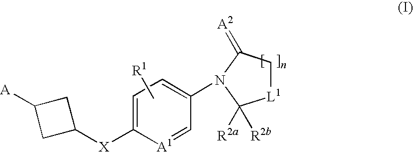

Generally, A¹ may be CH, C—F or N. In a particular embodiment, A¹ is CH.

In a specific embodiment, A² is oxygen.

In one embodiment X is O, S, NH or NCH₃. In a more specific embodiment X is O or S. In a further embodiment X is O.

In one embodiment, R¹ is hydrogen or halogen. In a very specific embodiment R¹ is hydrogen.

In one embodiment, R^{2a} is hydrogen, substituted or unsubstituted C₁₋₆ alkyl, a substituted or unsubstituted C₁₋₆-alkyl cycloalkyl, substituted or unsubstituted C₁₋₆-alkyl heterocycloalkyl, substituted or unsubstituted C₁₋₆-alkyl amino, substituted or unsubstituted C₁₋₆-alkyl ureido or substituted or unsubstituted C₁₋₆-alkyl aminocarbonyloxy as well as substituted or an unsubstituted aminocarbonyl.

In a more specific embodiment, R^{2a} is substituted or unsubstituted C₁₋₆-alkyl cycloalkyl, substituted or unsubstituted C₁₋₆-alkyl heterocycloalkyl, substituted or unsubstituted C₁₋₆-alkyl amino, substituted or unsubstituted aminocarbonyl, substituted or unsubstituted C₁₋₆-alkyl ureido, or substituted or unsubstituted C₁₋₆-alkyl aminocarbonyloxy.

In a further embodiment, R^{2a} is substituted or unsubstituted C₁₋₆-alkyl cycloalkyl, substituted or unsubstituted C₁₋₆-alkyl heterocycloalkyl or substituted or unsubstituted C₁₋₆-alkyl amino.

In another embodiment, R^{2a} is substituted or unsubstituted cyclohexylmethyl, substituted or unsubstituted piperidin-1-ylmethyl, substituted or unsubstituted morpholin-4-ylmethyl, substituted or unsubstituted pyrrolidin-1-ylmethyl, substituted or unsubstituted (ethyl)aminomethyl, substituted or unsubstituted [(pyrrolidin-1-ylcarbonyl)amino]methyl, substituted or unsubstituted [(methyl)(pyrrolidin-1-ylcarbonyl)amino]methyl, substituted or unsubstituted [(pyrrolidin-1-ylcarbonyl)oxy]methyl, substituted or unsubstituted [(piperidin-1-ylcarbonyl)oxy]methyl, substituted or unsubstituted [(morpholin-4-ylcarbonyl)oxy]methyl, substituted or unsubstituted (diethylamino)carbonyl, substituted or unsubstituted (cyclobutylamino)carbonyl, substituted or unsubstituted piperidin-1-ylcarbonyl, substituted or unsubstituted ethylaminocarbonyl, substituted or unsubstituted (methyl)(ethyl)amino-carbonyl, substituted or unsubstituted (phenyl)aminocarbonyl, substituted or unsubstituted [(phenyl)(methyl)amino]carbonyl, substituted or unsubstituted morpholin-4-ylcarbonyl and substituted or unsubstituted pyrrolidin-1-ylcarbonyl.

In a very specific embodiment, R^{2a} is cyclohexylmethyl, piperidin-1-ylmethyl, which may be further substituted, e.g. (4,4-difluoropiperidin-1-yl)methyl, morpholin-4-ylmethyl, pyrrolidin-1-ylmethyl, which may be further substituted, e.g. (3,3-difluoropyrrolidin-1-yl)methyl, [(2,2,2-trifluoroethyl)amino]methyl, [(pyrrolidin-1-ylcarbonyl)amino]methyl, [methyl(pyrrolidin-1-ylcarbonyl)amino]methyl, [(pyrrolidin-1-ylcarbonyl)oxy]methyl, [(piperidin-1-ylcarbonyl)oxy]methyl, which may be further substituted, e.g. {[(3,3-difluoropiperidin-1-yl)carbonyl]oxy}methyl or {[(4,4-difluoropiperidin-1-yl)carbonyl]oxy}-methyl, [(morpholin-4-ylcarbonyl)oxy]methyl, (diethylamino)carbonyl, (cyclobutylamino)-carbonyl, piperidin-1-ylcarbonyl, which may be further substituted, e.g. (4,4-difluoropiperidin-1-yl)carbonyl, [(2,2,2-trifluoroethyl)amino]-carbonyl, [methyl(2,2,2-trifluoroethyl)amino]carbonyl, [(4-fluorophenyl)amino]carbonyl, [(4-fluorophenyl)(methyl)-amino]carbonyl, morpholin-4-ylcarbonyl and (3,3-difluoropyrrolidin-1-yl)carbonyl.

In a more specific embodiment, R^{2a} is cyclohexylmethyl, piperidin-1-ylmethyl, (4,4-difluoropiperidin-1-yl)methyl, morpholin-4-ylmethyl, pyrrolidin-1-ylmethyl, (3,3-difluoropyrrolidin-1-yl)methyl, [(2,2,2-trifluoroethyl)amino]methyl, [(morpholin-4-ylcarbonyl)oxy]methyl, piperidin-1-ylcarbonyl, 4,4-difluoropiperidin-1-yl)carbonyl and morpholin-4-ylcarbonyl.

In an even more specific further embodiment, R^{2a} is piperidin-1-ylmethyl, (4,4-difluoropiperidin-1-yl)methyl, morpholin-4-ylmethyl, pyrrolidin-1-ylmethyl and (3,3-difluoropyrrolidin-1-yl)methyl.

In a particular embodiment, R^{2a} is piperidin-1-ylmethyl and pyrrolidin-1-ylmethyl, while R^{2b} is hydrogen.

In another particular embodiment, R^{2a} is (4,4-difluoropiperidin-1-yl)methyl, (3,3-difluoropyrrolidin-1-yl)methyl and morpholin-4-ylmethyl while R^{2b} is hydrogen.

In one particular embodiment R^{2b} is hydrogen.

In a further embodiment, A represents a group of formula —NR³R⁴ wherein R³ and R⁴ are independently substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted heterocycloalkyl, substituted or unsubstituted C₁₋₆-alkyl aryl, substituted or unsubstituted C₁₋₆-alkyl heteroaryl, substituted or unsubstituted C₁₋₆-alkyl cycloalkyl or substituted or unsubstituted C₁₋₆-alkyl heterocycloalkyl groups; or A is a 3 to 8 membered substituted or unsubstituted heterocycloalkyl linked to the cyclobutyl group via a nitrogen atom.

In a specific embodiment R³ is C₁₋₆ alkyl which may be substituted or unsubstituted, including C₁₋₆-alkyl cycloalkyl or C₁₋₆-alkyl aryl.

In a further specific embodiment R³ is a C₁₋₆ alkyl.

In one embodiment R⁴ is C₁₋₆ alkyl. Suitable examples include methyl or ethyl.

In another embodiment A is a group —NR³R⁴ wherein R³ and R⁴ are independently C₁₋₆ alkyl; or A is a 3 to 8 membered heterocycloalkyl linked to the cyclobutyl group via a nitrogen atom.

In a further particular embodiment A is a 3 to 8 membered heterocycloalkyl linked to the cyclobutyl group via a nitrogen atom.

In another particular embodiment A represents a 3 to 8 membered heterocycloalkyl selected from substituted or unsubstituted piperidin-1-yl, substituted or unsubstituted morpholin-4-yl, substituted or unsubstituted pyrrolidin-1-yl and substituted or unsubstituted piperazin-1-yl.

Typical examples for A include piperidin-1-yl, 4,4-difluoropiperidin-1-yl, morpholin-4-yl, pyrrolidin-1-yl, 3-(dimethylamino)pyrrolidin-1-yl, (3R)-3-(dimethylamino)-pyrrolidin-1-yl, 4-isopropylpiperazin-1-yl, 3-azepan-1-yl, 3-thiomorpholin-4-yl, 2-methylpyrrolidin-1-yl, (2S)-2-methylpyrrolidin-1-yl and (2R)-2-methylpyrrolidin-1-yl.

Typical examples for A include in particular piperidin-1-yl, 4,4-difluoropiperidin-1-yl, morpholin-4-yl, pyrrolidin-1-yl, 3-(dimethylamino)pyrrolidin-1-yl, (3R)-3-(dimethylamino)pyrrolidin-1-yl, 3-azepan-1-yl, 3-thiomorpholin-4-yl, 2-methylpyrrolidin-1-yl, (2S)-2-methylpyrrolidin-1-yl and (2R)-2-methylpyrrolidin-1-yl.

In a further embodiment A represents a 3 to 8 membered heterocycloalkyl selected from substituted or unsubstituted piperidin-1-yl, substituted or unsubstituted morpholin-4-yl, substituted or unsubstituted pyrrolidin-1-yl.

In one particular embodiment A is a 3 to 8 membered heterocycloalkyl selected from substituted or unsubstituted piperidin-1-yl, and substituted or unsubstituted pyrrolidin-1-yl.

In a more particular embodiment A is piperidin-1-yl, in a further embodiment A is 2-methylpyrrolidin-1-yl, (2R)-2-methylpyrrolidin-1-yl and (2S)-2-methylpyrrolidin-1-yl.

In one embodiment, R^{9a} is hydrogen or C₁₋₈ alkyl.

In one embodiment, R^{9b} is C₁₋₆-alkyl aryl or C₁₋₈ alkyl.

In one embodiment, the sum n+v+m+z is comprised between 1 and 5.

In one embodiment n is 0.

In another embodiment n is 1.

In one embodiment v is 0.

In another embodiment v is 1.

In one embodiment m is 0.

In one embodiment z is 1.

In one particular embodiment n is 1, v is 0, m is 0 and z is 1.

In another embodiment n is 1, v is 1, m is 0 and z is 1.

In a further embodiment n is 0, v is 1, m is equal to 0 and z is 1.

In one embodiment, the present invention relates to compounds of formula (I), geometrical isomers, enantiomers, diastereoisomers, pharmaceutically acceptable salts and all possible mixtures thereof,

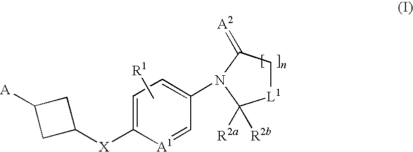

wherein

### A¹ is CH, C-halogen or N;

A² is oxygen or sulfur;
X is O, S, NH or N(C₁₋₄ alkyl);
R¹ is hydrogen or halogen, e.g. fluorine;
R^{2a} is hydrogen, substituted or unsubstituted C₁₋₆-alkyl, substituted or unsubstituted C₁₋₆-alkyl cycloalkyl, substituted or unsubstituted C₁₋₆-alkyl heterocycloalkyl, substituted or unsubstituted C₁₋₆-alkyl amino, aminocarbonyl, substituted or unsubstituted C₁₋₆-alkyl ureido or substituted or unsubstituted C₁₋₆-alkyl aminocarbonyloxy;
R^{2b} is hydrogen;
A is a group —NR³R⁴ wherein R³ and R⁴ are independently substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted heterocycloalkyl, substituted or unsubstituted C₁₋₆-alkyl aryl, substituted or unsubstituted C₁₋₆-alkyl heteroaryl, substituted or unsubstituted C₁₋₆-alkyl cycloalkyl or substituted or unsubstituted C₁₋₆-alkyl heterocycloalkyl groups; or A is a 3 to 8 membered substituted or unsubstituted heterocycloalkyl linked to the cyclobutyl group via a nitrogen atom;
L¹ is —(O)ᵥ—(CR^{9a}R^{9b})ₘ—(CH₂)_{z};
R^{9a} is hydrogen or unsubstituted C₁₋₈ alkyl;
R^{9b} is a C₁₋₆-alkyl aryl or unsubstituted C₁₋₈ alkyl;
n is an integer equal to 0 or 1;
v is an integer equal to 0 or 1;
m is an integer equal to 0 or 1;
z is an integer equal to 0, 1, 2 or 3;
and the sum n+v+m+z is comprised between 1 and 5.

In a second embodiment, the present invention relates to compounds of formula (I), geometrical isomers, enantiomers, diastereoisomers, pharmaceutically acceptable salts and all possible mixtures thereof,

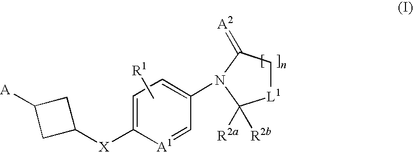

wherein

### A¹ is CH, C—F or N;

A² is oxygen;

### X is O, S, NH or NCH₃;

R¹ is hydrogen;
R^{2a} is substituted or unsubstituted C₁₋₆-alkyl cycloalkyl, substituted or unsubstituted C₁₋₆-alkyl heterocycloalkyl, substituted or unsubstituted C₁₋₆-alkyl amino, aminocarbonyl, substituted or unsubstituted C₁₋₆-alkyl ureido, or substituted or unsubstituted C₁₋₆-alkyl aminocarbonyloxy;
R^{2b} is hydrogen;
A is a group —NR³R⁴ wherein R³ and R⁴ are independently substituted or unsubstituted C₁₋₆ alkyl; or A is a 3 to 8 membered substituted or unsubstituted heterocycloalkyl linked to the cyclobutyl group via a nitrogen atom;
L¹ is —(O)ᵥ—(CR^{9a}R^{9b})ₘ—(CH₂)_{z};
R^{9a} is hydrogen or C₁₋₈ alkyl;
R^{9b} is a C₁₋₆-alkyl aryl or C₁₋₈ alkyl;
n is an integer equal to 0 or 1;
v is an integer equal to 0 or 1;
m is equal to 0;
z is equal to 1.

In a further embodiment, the present invention relates to compounds of formula (I), geometrical isomers, enantiomers, diastereoisomers, pharmaceutically acceptable salts and all possible mixtures thereof,

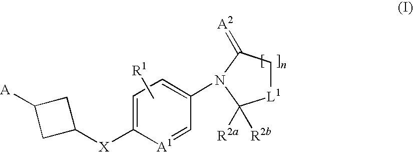

wherein

### A¹ is CH;

A² is oxygen;

### X is O;

R¹ is hydrogen;
R^{2a} is C₁₋₆-alkyl cycloalkyl, C₁₋₆-alkyl heterocycloalkyl or C₁₋₆-alkyl amino;
R^{2b} is hydrogen;
A is a 3 to 8 membered substituted or unsubstituted heterocycloalkyl linked to the cyclobutyl group via a nitrogen atom selected from substituted or unsubstituted piperidin-1-yl, substituted or unsubstituted morpholin-4-yl, substituted or unsubstituted pyrrolidin-1-yl and substituted or unsubstituted piperazin-1-yl;
L¹ is —(O)ᵥ—(CR^{9a}R^{9b})ₘ—(CH₂)_{z};
n is an integer equal to 0 or 1;
v is an integer equal to 0 or 1;
m is equal to 0;
z is equal to 1.

In another embodiment, the present invention relates to compounds of formula (I), geometrical isomers, enantiomers, diastereoisomers, pharmaceutically acceptable salts and all possible mixtures thereof,

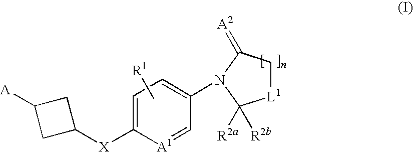

wherein

### A¹ is CH;

A² is oxygen;

### X is O;

R¹ is hydrogen;
R^{2a} is cyclohexylmethyl, piperidin-1-ylmethyl, (4,4-difluoropiperidin-1-yl)methyl, morpholin-4-ylmethyl, pyrrolidin-1-ylmethyl, (3,3-difluoropyrrolidin-1-yl)methyl, [(2,2,2-trifluoro-ethyl)amino]methyl, [(morpholin-4-ylcarbonyl)oxy]methyl, piperidin-1-ylcarbonyl, 4,4-difluoropiperidin-1-yl)carbonyl and morpholin-4-ylcarbonyl;
R^{2b} is hydrogen;
A is piperidin-1-yl, 2-methylpyrrolidin-1-yl, (2R)-2-methylpyrrolidin-1-yl and (2S)-2-methylpyrrolidin-1-yl;
L¹ is —(O)ᵥ—(CR^{9a}R^{9b})ₘ—(CH₂)_{z};
   n is an integer equal to 0 or 1;
   v is an integer equal to 0 or 1;
   m is equal to 0;
   z is equal to 1.

In a specific embodiment, the present invention relates to compounds of formula (I), geometrical isomers, enantiomers, diastereoisomers, pharmaceutically acceptable salts and all possible mixtures thereof,

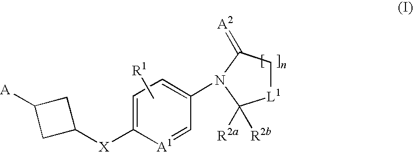

wherein

### A¹ is CH;

A² is oxygen;

### X is O;

R¹ is hydrogen;
R^{2a} is piperidin-1-ylmethyl or pyrrolidin-1-ylmethyl;
R^{2b} is hydrogen;
A is substituted or unsubstituted piperidin-1-yl, or substituted or unsubstituted pyrrolidin-1-yl;
L¹ is —(O)ᵥ—(CR^{9a}R^{9b})ₘ—(CH₂)_{z};
n is an integer equal to 0 or 1;
v is an integer equal to 0 or 1;
m is equal to 0;
z is equal to 1.

In a specific embodiment, the present invention relates to compounds of formula (I), geometrical isomers, enantiomers, diastereoisomers, pharmaceutically acceptable salts and all possible mixtures thereof,

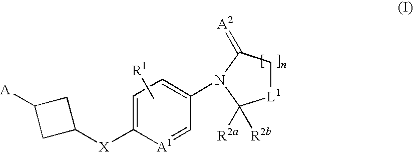

wherein

### A¹ is CH;

A² is oxygen;

### X is O;

R¹ is hydrogen;
R^{2a} is (4,4-difluoropiperidin-1-yl)methyl, (3,3-difluoropyrrolidin-1-yl)methyl and morpholin-4-ylmethyl;
R^{2b} is hydrogen;
A is substituted or unsubstituted piperidin-1-yl, or substituted or unsubstituted pyrrolidin-1-yl;
L¹ is —(O)ᵥ—(CR^{9a}R^{9b})ₘ—(CH₂)_{z};
n is an integer equal to 0 or 1;
v is an integer equal to 0 or 1;
m is equal to 0;
z is equal to 1.

In one aspect, the present invention relates to compounds of formula (Ia), geometrical isomers, enantiomers, diastereoisomers, pharmaceutically acceptable salts and all possible mixtures thereof,

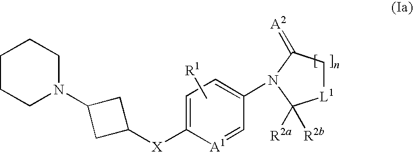

wherein A¹, A², X, R¹, R^{2a}, R^{2b}, L¹ and n are as herein defined.

Embodiments described hereinabove for A¹, A², X, R¹, R^{2a}, R^{2b}, L¹ and n in compounds of formula (I) also apply to A¹, A², X, R¹, R^{2a}, R^{2b}, L¹ and n in compounds of formula (Ia).

In another aspect, the present invention relates to compounds of formula (Ib), geometrical isomers, enantiomers, diastereoisomers, pharmaceutically acceptable salts and all possible mixtures thereof,

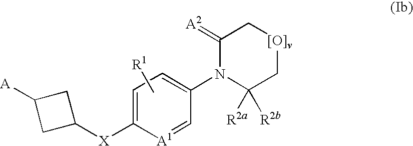

wherein A¹, A², X, R¹, R^{2a}, R^{2b}, A and v are as herein defined.

Embodiments described hereinabove for A¹, A², X, R¹, R^{2a}, R^{2b}, A and v in compounds of formula (I) also apply to A¹, A², X, R¹, R^{2a}, R^{2b}, A and v in compounds of formula (Ib).

In another aspect, the present invention relates to compounds of formula (Ic), geometrical isomers, enantiomers, diastereoisomers, pharmaceutically acceptable salts and all possible mixtures thereof,

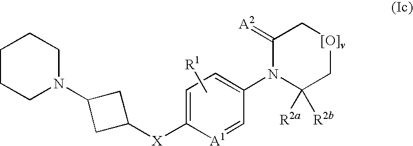

wherein A¹, A², X, R^{2a}, R^{2b} and v are as herein defined.

Embodiments described hereinabove for A¹, A², X, R¹, R^{2a}, R^{2b} and v in compounds of formula (I) also apply to A¹, A², X, R¹, R^{2a}, R^{2b} and v in compounds of formula (Ic).

In another aspect, the present invention relates to compounds of formula (Id), geometrical isomers, enantiomers, diastereoisomers, pharmaceutically acceptable salts and all possible mixtures thereof,

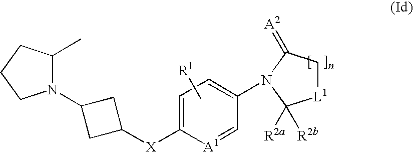

wherein A¹, A², X, R¹, R^{2a}, R^{2b}, L¹ and n are as herein defined.

Embodiments described hereinabove for A¹, A², X, R¹, R^{2a}, R^{2b} and v in compounds of formula (I) also apply to A¹, A², X, R¹, R^{2a}, R^{2b} and v in compounds of formula (Id).

According to a specific embodiment compounds of formulae (I), (Ia), (Ib), (Ic) and (Id), the A and X groups attached to the cyclobutyl in the A-cyclobutyl-X moiety are in trans configuration.

In a further aspect, the present invention relates to compounds of formula (I.I), geometrical isomers, enantiomers, diastereoisomers, pharmaceutically acceptable salts and all possible mixtures thereof,

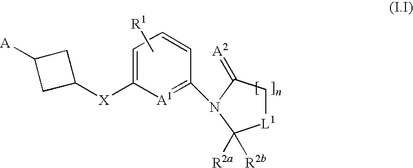

wherein A, X, A¹, A², R¹, R^{2a}, R^{2b}, L¹ and n are as herein defined.

Embodiments described hereinabove for A, X, A¹, A², R¹, R^{2a}, R^{2b}, L¹ and n in compounds of formula (I) also apply to A, X, A¹, A², R¹, R^{2a}, R^{2b}, L¹ and n in compounds of formula (I.I).

Examples of compounds according to the present invention are
(5S)-1-{4-[(trans-3-piperidin-1-ylcyclobutyl)oxy]phenyl}-5-(piperidin-1-ylmethyl)pyrrolidin-2-one;
(5S)-5-(cyclohexyl methyl)-4-{4-[(trans-3-piperidin-1-ylcyclobutyl)oxy]-phenyl}morpholin-3-one;
(5S)-5-[(4,4-difluoropiperidin-1-yl)methyl]-1-{4-[(trans-3-piperidin-1-ylcyclobutyl)oxy]-phenyl}pyrrolidin-2-one;
(5S)-5-(morpholin-4-ylmethyl)-1-{4-[(trans-3-piperidin-1-ylcyclobutyl)oxy]-phenyl}pyrrolidin-2-one;
(5S)-1-{4-[(trans-3-piperidin-1-ylcyclobutyl)oxy]phenyl}-5-(pyrrolidin-1-ylmethyl)pyrrolidin-2-one;
(5S)-1-{4-[(trans-3-piperidin-1-ylcyclobutyl)oxy]phenyl}-5-{[(2,2,2-trifluoroethyl)amino]methyl}-pyrrolidin-2-one;
(5S)-5-[(3,3-difluoropyrrolidin-1-yl)methyl]-1-{4-[(trans-3-piperidin-1-ylcyclobutyl)oxy]phenyl}-pyrrolidin-2-one;
(5S)-1-{3-[(trans-3-piperidin-1-ylcyclobutyl)oxy]phenyl}-5-(piperidin-1-ylmethyl)pyrrolidin-2-one;
5-[(4,4-difluoropiperidin-1-yl)methyl]-4-{4-[(trans-3-piperidin-1-ylcyclobutyl)oxy]phenyl}morpholin-3-one;
(5R)-5-(cyclohexylmethyl)-4-{4-[(trans-3-piperidin-1-ylcyclobutyl)oxy]phenyl}morpholin-3-one;
[(2S)-5-oxo-1-{4-[(trans-3-piperidin-1-ylcyclobutyl)oxy]phenyl}pyrrolidin-2-yl]methyl morpholine-4-carboxylate;
(4R)-4-[(4,4-difluoropiperidin-1-yl)methyl]-3-{4-[(trans-3-piperidin-1-ylcyclobutyl)oxy]phenyl}-1,3-oxazolidin-2-one;
(4R)-4-(morpholin-4-ylmethyl)-3-{4-[(trans-3-piperidin-1-ylcyclobutyl)oxy]phenyl}-1,3-oxazolidin-2-one;
(4R)-4-(cyclohexylmethyl)-3-{4-[(trans-3-piperidin-1-ylcyclobutyl)oxy]phenyl}-1,3-oxazolidin-2-one;
(4R)-3-{4-[(trans-3-piperidin-1-ylcyclobutyl)oxy]phenyl}-4-(piperidin-1-ylmethyl)-1,3-oxazolidin-2-one;
(5S)-5-(morpholin-4-ylmethyl)-1-{4-[(trans-3-piperidin-1-ylcyclobutyl)thio]phenyl}pyrrolidin-2-one;
(5S)-5-[(4,4-difluoropiperidin-1-yl)methyl]-1-{4-[(trans-3-piperidin-1-ylcyclobutyl)thio]-phenyl}pyrrolidin-2-one;
(4S)-4-(morpholin-4-ylcarbonyl)-3-{4-[(trans-3-piperidin-1-ylcyclobutyl)oxy]phenyl}-1,3-oxazolidin-2-one;
(4S)-4-(piperidin-1-ylcarbonyl)-3-{4-[(trans-3-piperidin-1-ylcyclobutyl)oxy]phenyl}-1,3-oxazolidin-2-one;
(4S)-4-[(4,4-difluoropiperidin-1-yl)carbonyl]-3-{4-[(trans-3-piperidin-1-ylcyclobutyl)oxy]phenyl}-1,3-oxazolidin-2-one;
(5S)-5-[(4,4-difluoropiperidin-1-yl)methyl]-1-(4-{[trans-3-(2-methylpyrrolidin-1-yl)cyclobutyl]oxy}phenyl)pyrrolidin-2-one;
(5S)-5-[(4,4-difluoropiperidin-1-yl)methyl]-1-(4-{[trans-3-((2R)-2-methylpyrrolidin-1-yl)cyclobutyl]oxy}phenyl)pyrrolidin-2-one; and
(5S)-5-[(4,4-difluoropiperidin-1-yl)methyl]-1-(4-{[trans-3-((2S)-2-methylpyrrolidin-1-yl)cyclobutyl]oxy}phenyl)pyrrolidin-2-one.

The compounds of the present invention are histamine H₃-receptor ligands. In one embodiment they are histamine H₃-receptor antagonists; in another embodiment they are histamine H₃-receptor inverse agonists.

In one embodiment, compounds of the present invention have particularly favorable drug properties, i.e. they have a good affinity to the H₃-receptor while having a low affinity towards other receptors or proteins; they have favorable pharmacokinetics and pharmacodynamics while having few side effect, e.g. toxicity such as cardiotoxicity. One of many methods known to determine the cardiovascular risk of drug compounds is to assess the binding of a test compound to hERG channels.

Compounds of the present invention display a low affinity on hERG channels (with a plC₅₀ of less than 6, preferably with a ratio (IC₅₀ hERG)/(IC₅₀H₃) greater than 1000.

The “pharmaceutically acceptable salts” according to the invention include therapeutically active, non-toxic acid salt forms which the compounds of formula (I) are able to form.

The acid addition salt form of a compound of formula (I′) or (I) that occurs in its free form as a base can be obtained by treating the free base with an appropriate acid such as an inorganic acid, for example, a hydrochloric, hydrobromic, sulfuric, nitric, phosphoric and the like; or an organic acid, such as, for example, acetic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, palmoic, and the like.

Conversely said salt forms can be converted into the free forms by treatment with an appropriate base.

Compounds of the formula (I′) or (I) and their salts can be in the form of a solvate, which is included within the scope of the present invention. Such solvates include for example hydrates, alcoholates and the like.

Many of the compounds of formula (I′) or (I) and some of their intermediates have at least one stereogenic center in their structure. This stereogenic center may be present in a R or a S configuration, said R and S notation is used in correspondence with the rules described in Pure Appl. Chem. 1976, 45, 11-30.

The invention also relates to all stereoisomeric forms such as enantiomeric and diastereomeric forms of the compounds of formula (I′) or (I) or mixtures thereof (including all possible mixtures of stereoisomers).

With respect to the present invention reference to a compound or compounds is intended to encompass that compound in each of its possible isomeric forms and mixtures thereof, unless the particular isomeric form is referred to specifically.

Compounds according to the present invention may exist in different polymorphic forms. Although not explicitly indicated in the above formula, such forms are included within the scope of the present invention.

The invention also includes within its scope pro-drug forms of the compounds of formula (I) and (I′) and its various sub-scopes and sub-groups.

The term “prodrug” as used herein includes compound forms which are rapidly transformed in vivo to the parent compound according to the invention, for example, by hydrolysis in blood. Prodrugs are compounds bearing groups which are removed by biotransformation prior to exhibiting their pharmacological action. Such groups include moieties which are readily cleaved in vivo from the compound bearing it, which compound after cleavage remains or becomes pharmacologically active. Metabolically cleavable groups form a class of groups well known to practitioners of the art. They include, but are not limited to such groups as alkanoyl (i.e. acetyl, propionyl, butyryl, and the like), unsubstituted and substituted carbocyclic aroyl (such as benzoyl, substituted benzoyl and 1- and 2-naphthoyl), alkoxycarbonyl (such as ethoxycarbonyl), trialklysilyl (such as trimethyl- and triethylsilyl), monoesters formed with dicarboxylic acids (such as succinyl), phosphate, sulfate, sulfonate, sulfonyl, sulfinyl and the like. The compounds bearing the metabolically cleavable groups have the advantage that they may exhibit improved bioavailability as a result of enhanced solubility and/or rate of absorption conferred upon the parent compound by virtue of the presence of the metabolically cleavable group. T. Higuchi and V. Stella, “Pro-drugs as Novel Delivery System”, Vol. 14 of the A.C.S. Symposium Series; “Bioreversible Carriers in Drug Design”, ed. Edward B. Roche, American Pharmaceutical Association and Pergamon Press, 1987.

Compounds of formula (I′) or (I) according to the invention may be prepared according to conventional methods known to the person skilled in the art of synthetic organic chemistry.

A. According to one embodiment, some compounds having the general formula (I) wherein A¹ is CH or C-halogen may be prepared by reaction of a compound of formula (II) with a compound of formula (III) according to the equation:

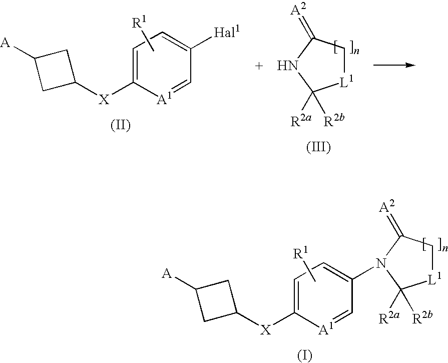

wherein A¹ is CH or C-halogen, Hal¹ is halogen, preferably bromine or iodine, and A, A², R¹, R^{2a}, R^{2b}, R⁴, R⁵ and L¹ have the same definitions as described above for compounds of formula I.

This reaction may be carried out using a catalyst such as copper iodide or palladium acetate, associated with a ligand such as 1,2-diamine (e.g. trans-1,2-diamineocyclohexane), a phosphine (e.g. 1,1′-bis(diphenylphosphino)ferrocene or 2-(dicyclohexylphosphino)-2′-(N,N-dimethylamino)-biphenyl) or an amino acid (e.g. glycine), in an inert solvent (such as dioxane, tetrahydrofuran, dimethylformamide or toluene), in the presence of a base (such as potassium phosphate or sodium tert-butylate), at a temperature ranging from 25° C. to 120° C. and under an inert atmosphere (argon or nitrogen).

Alternatively, this reaction may be performed according to the methodology described by Klapars A. et al. in J. Am. Chem. Soc. 2002, 124, 7421-7428. Compounds of formula (II) may be prepared according to any one of the following methods.

(a1) Compounds of formula (II) wherein A¹ is CH or C-halogen and X is O may be prepared by reaction of a compound of formula (IV) with a compound of formula (V) according to the equation:

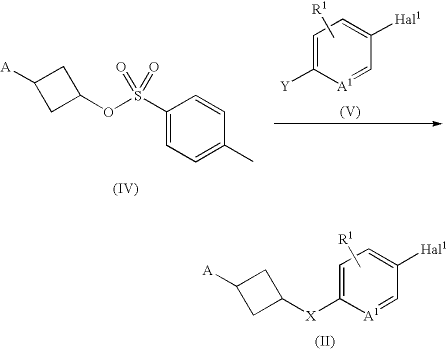

wherein A¹ is CH or C-halogen, X is O, Hall is bromine or iodine, Y is OH, A and R¹ having the same definitions as described above for compounds of formula I.

This reaction may be carried out using a base/solvent system such as sodium hydride/dimethylformamide, sodium hydride/dimethyl acetamide or potassium tert-butylate/dimethylsulfoxide, at a temperature ranging from 25° C. to 120° C., under an inert atmosphere (argon or nitrogen), or according to any conventional method known by the man skilled in the art.

Compounds of formula (IV) may be prepared by reaction of a compound of formula (VI) with p-toluenesulfonyl chloride according to the equation:

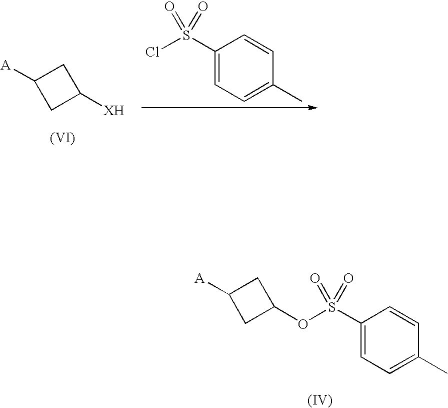

wherein X is O and A has the same definition as described above for compounds of formula I.

This reaction may be carried out using a base such as triethylamine or n-methylimidazole, in an inert solvent such as dichloromethane, at a temperature ranging from 0° C. to 25° C., under an inert atmosphere (argon or nitrogen), or according to any conventional method known by the man skilled in the art.

Compounds of formula (V) are commercially available or may be prepared according to any conventional method known to the person skilled in the art.

Compound of formula (VI) wherein X is O may be commercially available or prepared from a compound of formula (VII), according to the equation:

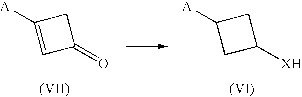

wherein X is O and A has the same definition as described above for compounds of formula I

This reaction may be carried out using a reductive agent such as sodium borohydride, in a protic solvent such as ethanol, at a temperature ranging from 0° C. to 60 C., under an inert atmosphere (argon or nitrogen), or according to any conventional method known by the man skilled in the art.

Compound of formula (VII) may be commercially available or prepared from cyclobutane-1,3-dione by reaction with an amine of formula AH, according to the equation:

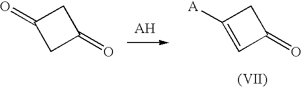

wherein A has the same definition as described above for compounds of formula I. Examples of AH are piperidine, 4,4-difluoropiperidine, morpholine, pyrrolidine, 2-methylpyrrolidine, (2R)-2-methylpyrrolidine, (2S)-2-methylpyrrolidine, (3R)-3-(dimethylamino)pyrrolidine, 4-iopropylpiperazine, azepane and thiomorpholine.

This reaction may be carried out in an inert solvent such as dioxane, at a temperature ranging from 0° C. to 30° C., under an inert atmosphere (argon or nitrogen), or according to any conventional method known by the man skilled in the art. Cyclobutan-1,3-dione is commercially available or may be prepared according to any conventional method known to the person skilled in the art.

(a2) Compounds of formula (II) wherein A¹ is CH or C-halogen and X is S may be prepared by reaction of a compound of formula (V) with compound of formula (VI) according to the equation:

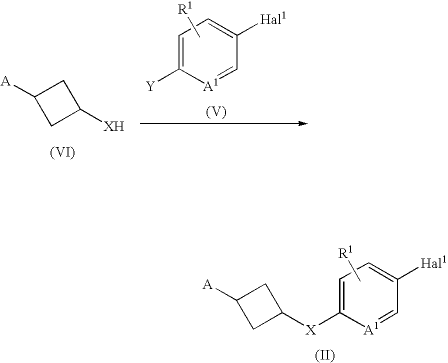

wherein A¹ is CH or C-halogen, X is S, Hall is bromine or iodine, Y is fluorine, A and R¹ having the same definitions as described above for compounds of formula I.

This reaction may be carried out according to the method described by Kwong, F. Y. and Buchwald, S. L. in Org. Lett. 2002, 4, 3517-3520, i.e., using a base (e.g., potassium carbonate), a catalyst (e.g., copper iodide), in a protic solvent (e.g., 2-propanol), in the presence of a co-solvent (e.g. ethylene glycol), at a temperature ranging from 25° C. to 100° C., under an inert atmosphere (argon or nitrogen). Alternatively, this reaction may be performed according to any other conventional method known by the man skilled in the art.

Compounds of formula (VI) wherein X is S may be prepared from compound of formula (IV) according to the equation:

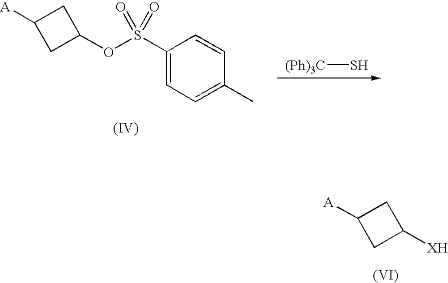

wherein X is S and A has the same definition as described above for compounds of formula I

This reaction may be carried out according to the method described by Oh, C.-H. and Sho, J.-H. in Eur. J. Med. Chem. 2006, 41, 50-55, i.e., using triphenylmethylthiol in the presence of a base (e.g., sodium hydride) and an inert solvent (e.g., dimethylformamide), at a temperature ranging from 0° C. to 100° C., under an inert atmosphere (argon or nitrogen), followed by deprotection of the triphenylmethyl group using a trifluoroacetic acid/triethylsilane reductive system. Alternatively, this reaction scheme may be performed according to any other conventional method known by the man skilled in the art.

(a3) Compounds of formula (II) wherein A¹ is CH or C-halogen and X is NH or N(C₁₋₆ alkyl) may be prepared by reaction of a compound of formula (VII) with a compound of formula (V′) according to the equation:

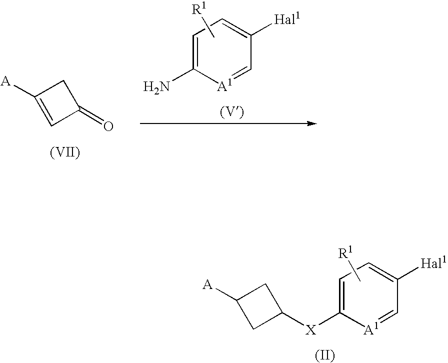

wherein A¹ is CH or C-halogen, X is NH or N(C₁₋₆ alkyl), Hall is bromine or iodine, Y is chlorine, fluorine or trifluoromethylsulfonate, A and R¹ having the same definitions as described above for compounds of formula I.

This reaction may be carried out using a reducing agent, such as sodium cyanoborohydride, in acetic acid and at room temperature, or according to any other conventional method known by the man skilled in the art.

Compounds of formula (III) may be commercially available or prepared according to any one of the following methods.

(a.4) Compounds of formula (III) wherein R^{2a} is C₁₋₆-alkyl amino and R^{2b} is hydrogen may be obtained by the reaction of a compound of formula (IX) with a primary or secondary amine, preferably a cyclic amine, according to the equation:

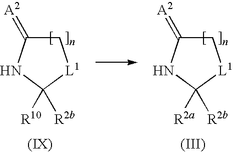

wherein R¹⁰ is a C₁₋₆-alkyl substituted by a leaving group, R^{2a} is C₁₋₆-alkyl amino, R^{2b} is hydrogen, and L¹, A² and n have the same definitions as described above for compounds of formula (I).

The term “leaving group”, as used herein, has the same meaning by the person skilled in the art as defined in “Advanced Organic Chemistry: reactions, mechanisms and structure—Third Edition by Jerry March, John Wiley and Sons Ed.; 1985 page 179”.

Examples of leaving groups are sulfonates, for example methylsulfonate, and halogens, for example chlorine, bromine or iodine.

The term “sulfonate”, as used herein, represents a group of formula —O—SO₂—R^{e} wherein R^{e} is C₁₋₄ alkyl or aryl as defined above in the specifications.

This reaction may be carried out according to the method described by Kenda, B. et al. in J. Med. Chem. 2004, 47, 530-549, or according to any conventional method known to the person skilled in the art.

Amines of formula G¹-H may be commercially available or may be prepared according to any conventional method known to the person skilled in the art.

(a.5) Compounds of formula (III) wherein R^{2a} and R^{2b} are linked together to form a C₃₋₈ cycloalkyl or a 3-8 membered heterocycloalkyl may be prepared according to the methods described in Cignarella, G. et al. in J. Heterocycl. Chem. 1993, 30, 1357-1359; by Smith, Paul W. et al. in J. Med. Chem. 1995, 38, 3772-3779; or according to any conventional method known to the person skilled in the art.

(a.6) Compounds of formula (III) wherein A² is O, L¹ is —(O)ᵥ—(CR^{9a}R^{9b})ₘ—(CH₂)_{z}—, v is 1 and n is 0 may be obtained by cyclisation of the corresponding amino-alcohol of formula (X) according to the equation:

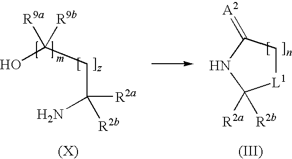

wherein A² is O, L¹ is —(O)ᵥ—(CR^{9a}R^{9b})ₘ—(CH₂)_{z}—, v is 1 and n is 0, R^{2a}, R^{2b}, m, R^{9a}, R^{9b} and z having the same definitions as described above for compounds of formula (I). This reaction may be performed in the presence of carbonic acid bis-trichloromethyl ester (or triphosgene) according to the method described by Ding, K. et al. in Tetrahedron Lett. 2004, 45, 1027-1029; or in the presence of carbonic acid diethyl ester according to the method described by Tomioka, K. in Tetrahedron 1993, 49, 1891-1900; or according to any other conventional method known to the person skilled in the art.

Compounds of formula (X) are commercially available or may be prepared according to any conventional method known to the person skilled in the art.

(a.7) Compounds of formula (III) wherein A² is 0, L¹ is —(O)ᵥ—(CR^{9a}R^{9b})ₘ—(CH₂)_{z}—, v is 0, m is 1 and n is 0 may be obtained using the procedure described by Davies, S. B. et al. in Tetrahedron Asym. 2002, 13, 647-658.

(a.8) Compounds of formula (III) wherein A² is 0, L¹ is —(O)ᵥ—(CR^{9a}R^{9b})ₘ—(CH₂)_{z}— and v is 0 may be obtained by cyclisation of the corresponding amino-acid or an amino-ester of formula (XI) according to the equation:

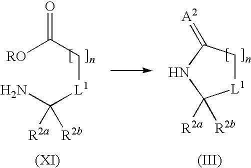

wherein R is hydrogen or a C₁₋₄ alkyl, A² is 0, L¹ is —(O)ᵥ—(CR^{9a}R^{9b})ₘ—(CH₂)_{z}— and v is 0, n, R^{2a} and R^{2b} having the same definitions as described above for compounds of formula (I).

This reaction may be performed according to the method described by Lopez-Garcia, M. et al. in J. Org. Chem. 2003, 68, 648-651, or according to any other conventional method known to the person skilled in the art.

Compounds of formula (XI) are commercially available or may be prepared from the corresponding nitro-ester by hydrogenolysis of the nitro group according to any conventional method known to the person skilled in the art.

(a.9) Compounds of formula (III) wherein A² is O, n is 0 or 1, L¹ is —(O)ᵥ—(CR^{9a}R^{9b})ₘ—(CH₂)_{z}— and v is 1 may be obtained by cyclisation of the corresponding compound of formula (XII), according to the following equation:

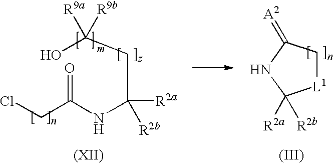

wherein A² is O, L¹ is —(O)ᵥ—(CR^{9a}R^{9b})ₘ—(CH₂)_{z}— and v is 1.

In particular, when n is 1, this reaction may be performed using a base such as potassium tert-butylate in a protic solvent, such as 2-propanol, at a temperature ranging from 0° C. to 100° C.; or using sodium hydride in tetrahydrofuran, as described by Norman et al in J. Org. Chem. 1996, 61, 4990-4998, or according to any other conventional method known to the person skilled in the art.

Compounds of formula (XII) may be obtained by reaction compound of formula (X) with chloroacetyl chloride in the presence of a base (e.g., potassium carbonate), in an inert solvent such as tetrahydrofuran or a mixture of tetrahydrofuran and water, at a temperature ranging from 0° C. to 100° C., preferably at room temperature; or according to any other conventional method known to the person skilled in the art.

(a.10) Compounds of formula (III) wherein A² is O, n is 0, L1 is —OCH₂—, R^{2b} is H and R^{2a} is substituted aminocarbonyl can be prepared from commercially available 4-carbethoxyoxazolidin-2-one by conventional methods known to the man skilled in the art.

B. According to another embodiment, some compounds having the general formula (I) wherein A¹ is N may be prepared by reaction of a compound of formula (XIII) with a compound of formula (VI) according to the equation:

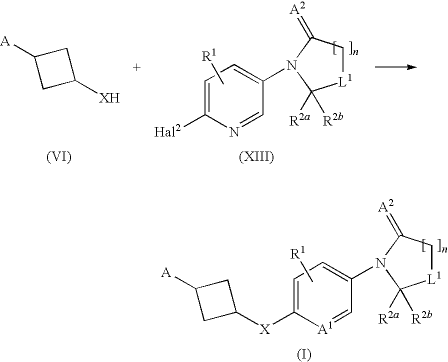

wherein A¹ is N, Hal² is halogen, preferably fluorine or chlorine, and A, X, A², R¹, R^{2a}, R^{2b}, n and L¹ have the same definitions as described above for compounds of formula I.

This reaction may be performed in the presence of a base (e.g., potassium tert-butylate, cesium carbonate or sodium hydride), in a solvent, (e.g., dimethylformamide or tetrahydrofuran), in the presence of a palladium- or a copper-based catalyst together with a ligand (e.g., 1,1′-bis(diphenylphosphino)ferrocene or 2-(dicyclohexylphosphino)-2′-(N,N-dimethylamino)-biphenyl), at a temperature ranging from 25° C. to 120° C., according to methods described by Penning, T. D. et al. in J. Med. Chem. 2000, 43, 721-735 or Westland, R. D. et al. in J. Med. Chem. 1973, 16, 319-327.

Compounds of formula (XIII) wherein A² is 0, L¹ is —(O)ᵥ—(CR^{9a}R^{9b})ₘ—(CH₂)_{z}— and v is 1 may be obtained by reaction of the compound of formula (XIV) with a compound of formula (III) according to the equation:

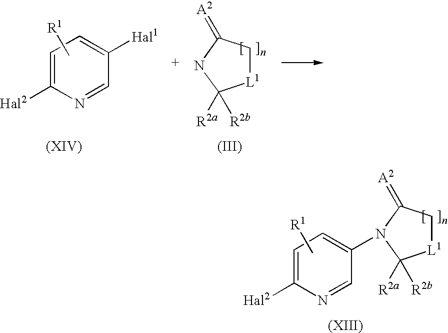

wherein Hal¹ is halogen, preferably bromine or iodine, Hal² is halogen, preferably fluorine or chlorine, and A², R¹, R^{2a}, R^{2b}, n and L¹ have the same definitions as described above for compounds of formula I.

This reaction may be carried out using a catalyst such as copper iodide or palladium acetate, associated with a ligand such as 1,2-diamine (e.g. trans-1,2-diamineocyclohexane), a phosphine (e.g. 1,1′-bis(diphenylphosphino)ferrocene or 2-(dicyclohexylphosphino)-2′-(N,N-dimethylamino)-biphenyl) or an amino acid (e.g. glycine), in an inert solvent (such as dioxane, tetrahydrofuran, dimethylformamide or toluene), in the presence of a base (such as potassium phosphate or sodium tert-butylate), at a temperature ranging from 25° C. to 120° C. and under an inert atmosphere (argon or nitrogen).

Alternatively, this reaction may be performed according to the methodology described by Klapars A. et al. in J. Am. Chem. Soc. 2002, 124, 7421-7428.

Compounds of formula (XIV) are commercially available or may be prepared according to any conventional methods known to the person skilled in the art.

Compounds of formula (I.I) can be made in a similar manner to (I).

In a further embodiment, the present invention relates to synthetic intermediates of formula (II), geometrical isomers, enantiomers, diastereoisomers:

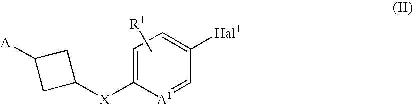

wherein A, A¹, X, and R¹ are as above defined and Hal¹ is a halogen.

Compounds of formula (II) are particularly useful for the synthesis of a compound of formula (I).

Examples of synthetic intermediates in particular of formula (II) are
3-(2-methylpyrrolidin-1-yl)cyclobut-2-en-1-one;
3-morpholin-4-ylcyclobut-2-en-1-one;
3-(4-isopropylpiperazin-1-yl)cyclobut-2-en-1-one;
3-(4,4-difluoropiperidin-1-yl)cyclobut-2-en-1-one;
3-azepan-1-ylcyclobut-2-en-1-one;
3-[(3R)-3-(dimethylamino)pyrrolidin-1-yl]cyclobut-2-en-1-one;
3-thiomorpholin-4-ylcyclobut-2-en-1-one;
cis-3-piperidin-1-ylcyclobutanol;
cis-3-(2-methylpyrrolidin-1-yl)cyclobutanol;
cis-3-morpholin-4-ylcyclobutanol;
cis-3-(4-isopropylpiperazin-1-yl)cyclobutanol;
cis-3-(4,4-difluoropiperidin-1-yl)cyclobutanol;
cis-3-pyrrolidin-1-ylcyclobutanol;
cis-3-azepan-1-ylcyclobutanol;
cis-3-[(3R)-3-(dimethylamino)pyrrolidin-1-yl]cyclobutanol;
cis-3-thiomorpholin-4-ylcyclobutanol;
cis-3-piperidin-1-ylcyclobutyl 4-methylbenzenesulfonate;
cis-3-(2-methylpyrrolidin-1-yl)cyclobutyl 4-methylbenzenesulfonate;
cis-3-(2-methylpyrrolidin-1-yl)cyclobutyl 4-bromobenzenesulfonate;
cis-3-morpholin-4-ylcyclobutyl 4-methylbenzenesulfonate;
cis-3-(4-isopropylpiperazin-1-yl)cyclobutyl 4-methylbenzenesulfonate;
cis-3-(4,4-difluoropiperidin-1-yl)cyclobutyl 4-methylbenzenesulfonate;
cis-3-pyrrolidin-1-ylcyclobutyl 4-methylbenzenesulfonate;
cis-3-azepan-1-ylcyclobutyl 4-methylbenzenesulfonate;
cis-3-[(3R)-3-(dimethylamino)pyrrolidin-1-yl]cyclobutyl 4-methylbenzenesulfonate;
cis-3-thiomorpholin-4-ylcyclobutyl 4-methylbenzenesulfonate;
1-[trans-3-(4-bromophenoxy)cyclobutyl]piperidine;
1-[trans-3-(3-bromophenoxy)cyclobutyl]piperidine;
1-[trans-3-(4-iodophenoxy)cyclobutyl]piperidine;
1-[trans-3-(4-iodophenoxy)cyclobutyl]-2-methylpyrrolidine;
(2R)-1-[trans-3-(4-iodophenoxy)cyclobutyl]-2-methylpyrrolidine;
(2S)-1-[trans-3-(4-iodophenoxy)cyclobutyl]-2-methylpyrrolidine;
(5S)-5-[(3,3-difluoropyrrolidin-1-yl)methyl]pyrrolidin-2-one;
2-chloro-N-[(1S)-2-cyclohexyl-1-(hydroxymethyl)ethyl]acetamide;
(5S)-5-(cyclohexylmethyl)morpholin-3-one;
(5R)-5-(cyclohexylmethyl)morpholin-3-one;
[(2S)-5-oxopyrrolidin-2-yl]methyl morpholine-4-carboxylate;
(4R)-4-(morpholin-4-ylmethyl)-1,3-oxazolidin-2-one;
propan-2-yl 5-oxomorpholine-3-carboxylate;
5-(hydroxymethyl)morpholin-3-one;
(5-oxomorpholin-3-yl)methyl 4-methylbenzenesulfonate;
5-[(4,4-difluoropiperidin-1-yl)methyl]morpholin-3-one;
1-[trans-3-(tritylsulfanyl)cyclobutyl]piperidine;
trans-3-[butyl(ethyl)amino]cyclobutanethiol;
1-{trans-3-[(4-iodophenyl)sulfanyl]cyclobutyl}piperidine;
tert-butyl (4S)-2,2-dimethyl-4-(morpholin-4-ylcarbonyl)-1,3-oxazolidine-3-carboxylate;
tert-butyl (4S)-2,2-dimethyl-4-(piperidin-1-ylcarbonyl)-1,3-oxazolidine-3-carboxylate;
tert-butyl (4S)-4-[(4,4-difluoropiperidin-1-yl)carbonyl]-2,2-dimethyl-1,3-oxazolidine-3-carboxylate;
(2S)-2-amino-1-(4,4-difluoropiperidin-1-yl)-3-hydroxypropan-1-one hydrochloride;
(4S)-4-(morpholin-4-ylcarbonyl)-1,3-oxazolidin-2-one;
(4S)-4-(piperidin-1-ylcarbonyl)-1,3-oxazolidin-2-one; and
(4S)-4-[(4,4-difluoropiperidin-1-yl)carbonyl]-1,3-oxazolidin-2-one.

It has now been found that compounds of formula (I) and (I′) according to the present invention and their pharmaceutically acceptable salts are useful in a variety of medical disorders.

For example, the compounds according to the invention are useful for the treatment and prevention of diseases or pathological conditions of the central nervous system including mild-cognitive impairments, Alzheimer's disease, learning and memory disorders, cognitive disorders, attention deficit disorder, attention-deficit hyperactivity disorder, Parkinson's disease, schizophrenia, dementia, depression, epilepsy, seizures, convulsions, sleep/wake and arousal/vigilance disorders such as hypersomnia and narcolepsy, pain and/or obesity.

Furthermore, compounds according to the invention alone or in combination with an antiepileptic drug (AED) may be useful in the treatment of epilepsy, seizure or convulsions. It is known from literature that the combination of H₃-receptor ligands with an AED may produce additive synergistic effects on efficacy with reduced side-effects such as decreased vigilance, sedation or cognitive problems.

Furthermore, compounds of the present invention alone or in combination with a histamine H₁ antagonist may also be used for the treatment of upper airway allergic disorders.

In a particular embodiment of the present invention, compounds of the present invention alone or in combination with muscarinic receptor ligands and particularly with a muscarinic M₂ antagonist, may be useful for the treatment of cognitive disorders, Alzheimer's disease, and attention-deficit hyperactivity disorder.

Particularly, compounds of general formula (I) or (I′) displaying NO-donor properties, alone or in combination with a nitric oxide (NO) releasing agent may be useful in the treatment of cognitive dysfunctions.

Compounds of general formula (I) or (I′) may also be used in the treatment and prevention of multiple sclerosis (MS).

Usually, compounds of general formula (I) may be used in the treatment and prevention of all types of cognitive-related disorders.

In one embodiment, compounds of general formula (I) or (I′) may be used for the treatment and prevention of cognitive dysfunctions in diseases such as mild cognitive impairment, dementia, Alzheimer's disease, Parkinson's disease, Down's syndrome as well as for the treatment of attention-deficit hyperactivity disorder.

In another embodiment, compounds of general formula (I) or (I′) may also be used for the treatment and prevention of psychotic disorders, such as schizophrenia; or for the treatment of eating disorders, such as obesity; or for the treatment of inflammation and pain; or for the treatment of anxiety, stress and depression; or for the treatment of cardiovascular disorders, for example, myocardial infarction; or for the treatment and prevention of multiple sclerosis (MS).

In one embodiment, compounds of formula (I) or (I′) according to the present invention may be used as a medicament.

In a further embodiment, the present invention concerns the use of a compound of formula (I) or (I′) or a pharmaceutically acceptable salt thereof or of a pharmaceutical composition comprising an effective amount of said compound for the treatment and prevention of mild-cognitive impairment, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation, pain or multiple sclerosis.

In another embodiment, the present invention concerns the use of a compound of formula (I) or (I′) or a pharmaceutically acceptable salt thereof or a pharmaceutical composition comprising an effective amount of said compound for the manufacture of a medicament for the treatment of cognitive dysfunctions in diseases such as mild cognitive impairment, dementia, Alzheimer's disease, Parkinson's disease, Down's syndrome as well as for the treatment of attention-deficit hyperactivity disorder.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 3 to 3000 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The term “cognitive disorders” as used herein refers to disturbances of cognition, which encompasses perception, learning and reasoning or in other terms the physiological (mental/neuronal) process of selectively acquiring, storing, and recalling information.

The term “attention-deficit hyperactivity disorder” (ADHD) as used herein refers to a problem with inattentiveness, over-activity, impulsivity, or a combination of these. For these problems to be diagnosed as ADHD, they must be out of the normal range for the child's age and development. The term “attention-deficit disorder” (ADD) is also commonly used for the same disorder.

The term “Alzheimer's disease” (AD) as used herein refers to a progressive, neurodegenerative disease characterized in the brain by abnormal clumps (amyloid plaques) and tangled bundles of fibers (neurofibrillary tangles) composed of misplaced proteins. Age is the most important risk factor for AD; the number of people with the disease doubles every 5 years beyond age 65. Three genes have been discovered that cause early onset (familial) AD. Other genetic mutations that cause excessive accumulation of amyloid protein are associated with age-related (sporadic) AD. Symptoms of AD include memory loss, language deterioration, impaired ability to mentally manipulate visual information, poor judgment, confusion, restlessness, and mood swings. Eventually AD destroys cognition, personality, and the ability to function. The early symptoms of AD, which include forgetfulness and loss of concentration, are often missed because they resemble natural signs of aging.

The term “Parkinson's disease” (PD) as used herein refers to a group of conditions called motor system disorders, which are the result of the loss of dopamine-producing brain cells. The four primary symptoms of PD are tremor, or trembling in hands, arms, legs, jaw, and face; rigidity, or stiffness of the limbs and trunk; bradykinesia, or slowness of movement; and postural instability, or impaired balance and coordination. As these symptoms become more pronounced, patients may have difficulty walking, talking, or completing other simple tasks. PD usually affects people over the age of 50. Early symptoms of PD are subtle and occur gradually. In some people the disease progresses more quickly than in others. As the disease progresses, the shaking, or tremor, which affects the majority of PD patients may begin to interfere with daily activities. Other symptoms may include depression and other emotional changes; difficulty in swallowing, chewing, and speaking; urinary problems or constipation; skin problems; and sleep disruptions.

The term “Down's syndrome” as used herein refers to a chromosome abnormality, usually due to an extra copy of the 21^{st} chromosome. This syndrome, usually but not always results in mental retardation and other conditions. The term “mental retardation” refers to a below-average general intellectual function with associated deficits in adaptive behavior that occurs before age 18.

The term “mild-cognitive impairment” as used herein refers to a transitional stage of cognitive impairment between normal aging and early Alzheimer's disease. It refers particularly to a clinical state of individuals who are memory impaired but are otherwise functioning well and do not meet clinical criteria for dementia.

The term “obesity” as used herein refers to a body mass index (BMI) which is greater than 30 kg/m².

The term “dementia” as used herein refers to a group of symptoms involving progressive impairment of brain function. American Geriatrics Society refers to dementia as a condition of declining mental abilities, especially memory. The person will have problems doing things he or she used to be able to do, like keep the check book, drive a car safely, or plan a meal. He or she will often have problems finding the right words and may become confused when given too many things to do at once. The person with dementia may also change in personality, becoming aggressive, paranoid, or depressed.

The term “schizophrenia” as used herein refers to a group of psychotic disorders characterized by disturbances in thought, perception, attention, affect, behavior, and communication that last longer than 6 months. It is a disease that makes it difficult for a person to tell the difference between real and unreal experiences, to think logically, to have normal emotional responses to others, and to behave normally in social situations.

The term “anxiety” as used herein refers to a feeling of apprehension or fear. Anxiety is often accompanied by physical symptoms, including twitching or trembling, muscle tension, headaches, sweating, dry mouth, difficulty swallowing and/or abdominal pain.

The term “narcolepsy” as used herein refers to a sleep disorder associated with uncontrollable sleepiness and frequent daytime sleeping.

The term “depression” as used herein refers to a disturbance of mood and is characterized by a loss of interest or pleasure in normal everyday activities. People who are depressed may feel “down in the dumps” for weeks, months, or even years at a time. Some of the following symptoms may be symptoms of depression: persistent sad, anxious, or “empty” mood; feelings of hopelessness, pessimism; feelings of guilt, worthlessness, helplessness; loss of interest or pleasure in hobbies and activities that were once enjoyed, including sex; decreased energy, fatigue, being “slowed down”; difficulty concentrating, remembering, making decisions; insomnia, early-morning awakening, or oversleeping; appetite and/or weight loss or overeating and weight gain; thoughts of death or suicide; suicide attempts; restlessness, irritability; persistent physical symptoms that do not respond to treatment, such as headaches, digestive disorders, and chronic pain.

The term “epilepsy” as used herein refers a brain disorder in which clusters of nerve cells, or neurons, in the brain sometimes signal abnormally. In epilepsy, the normal pattern of neuronal activity becomes disturbed, causing strange sensations, emotions, and behavior or sometimes convulsions, muscle spasms, and loss of consciousness. Epilepsy is a disorder with many possible causes. Anything that disturbs the normal pattern of neuron activity—from illness to brain damage to abnormal brain development—can lead to seizures. Epilepsy may develop because of an abnormality in brain wiring, an imbalance of nerve signaling chemicals called neurotransmitters, or some combination of these factors. Having a seizure does not necessarily mean that a person has epilepsy. Only when a person has had two or more seizures is he or she considered to have epilepsy.

The term “seizure” as used herein refers to a transient alteration of behaviour due to the disordered, synchronous, and rhythmic firing of populations of brain neurones.

The term “migraine” as used herein means a disorder characterised by recurrent attacks of headache that vary widely in intensity, frequency, and duration. The pain of a migraine headache is often described as an intense pulsing or throbbing pain in one area of the head. It is often accompanied by extreme sensitivity to light and sound, nausea, and vomiting. Some individuals can predict the onset of a migraine because it is preceded by an “aura,” visual disturbances that appear as flashing lights, zig-zag lines or a temporary loss of vision. People with migraine tend to have recurring attacks triggered by a lack of food or sleep, exposure to light or hormonal irregularities (only in women). Anxiety, stress, or relaxation after stress can also be triggers. For many years, scientists believed that migraines were linked to the dilation and constriction of blood vessels in the head. Investigators now believe that migraine is caused by inherited abnormalities in genes that control the activities of certain cell populations in the brain. The International Headache Society (IHS, 1988) classifies migraine with aura (classical migraine) and migraine without aura (common migraine) as the major types of migraine.

The term “multiple sclerosis” (MS) as used herein is a chronic disease of the central nervous system in which gradual destruction of myelin occurs in patches throughout the brain or spinal cord or both, interfering with the nerve pathways. As more and more nerves are affected, a patient experiences a progressive interference with functions that are controlled by the nervous system such as vision, speech, walking, writing, and memory.

Activity in any of the above-mentioned indications can of course be determined by carrying out suitable clinical trials in a manner known to a person skilled in the relevant art for the particular indication and/or in the design of clinical trials in general.

For treating diseases, compounds of formula (I) or (I′) or their pharmaceutically acceptable salts may be employed at an effective daily dosage and administered in the form of a pharmaceutical composition.

Therefore, another embodiment of the present invention concerns a pharmaceutical composition comprising an effective amount of a compound of formula (I) or a pharmaceutically acceptable salt thereof in combination with a pharmaceutically acceptable diluent or carrier.

To prepare a pharmaceutical composition according to the invention, one or more of the compounds of formula (I) or (I′) or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral or intranasal.

Pharmaceutical compositions comprising compounds according to the invention can, for example, be administered orally, parenterally, i.e., intravenously, intramuscularly or subcutaneously, intrathecally, by inhalation or intranasally.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula (I) or (I′) in compositions for oral administration is at least 0.5% by weight and can be up to 80% by weight with respect to the total weight of the composition.

For the preferred oral compositions, the daily dosage is in the range 3 to 3000 milligrams (mg) of compounds of formula (I) or (I′).

In compositions for parenteral administration, the quantity of compound of formula (I) or (I′) present is at least 0.5% by weight and can be up to 33% by weight with respect to the total weight of the composition. For the preferred parenteral compositions, the dosage unit is in the range 3 mg to 3000 mg of compounds of formula (I) or (I′).

The daily dose can fall within a wide range of dosage units of compound of formula (I) or (I′) and is generally in the range 3 to 3000 mg. However, it should be understood that the specific doses can be adapted to particular cases depending on the individual requirements, at the physician's discretion.

The following examples illustrate how the compounds covered by formula (I) or (I′) may be synthesized. They are provided for illustrative purposes only and are not intended, nor should they be construed, as limiting the invention in any manner. Those skilled in the art will appreciate that routine variations and modifications of the following examples can be made without exceeding the spirit or scope of the invention.

NMR spectra are recorded on a BRUKER AVANCE 400 NMR Spectrometer fitted with a Linux workstation running XWIN NMR 3.5 software and a 5 mm inverse 1H/BB probehead, or BRUKER DRX 400 NMR fitted with a SG Fuel running XWIN NMR 2.6 software and a 5 mm inverse geometry ¹H/¹³C/¹⁹F triple probehead. The compound is studied in d₆-dimethylsulfoxide (or d₃-chloroform) solution at a probe temperature of 313 K or 300 K and at a concentration of 10 mg/ml. The instrument is locked on the deuterium signal of d₆-dimethylsulfoxide (or d₃-chloroform). Chemical shifts are given in ppm downfield from TMS (tetramethylsilane) taken as internal standard.

HPLC analyses are performed using one of the following systems:
an Agilent 1100 series HPLC system mounted with an INERTSIL ODS 3 C18, DP 5 μm, 250×4.6 mm column. The gradient runs from 100% solvent A (acetonitrile, water, phosphoric acid (5/95/0.001, v/v/v)) to 100% solvent B (acetonitrile, water, phosphoric acid (95/5/0.001, v/v/v)) in 6 min with a hold at 100% B of 4 min. The flow rate is set at 2.5 ml/min. The chromatography is carried out at 35° C.
a HP 1090 series HPLC system mounted with a HPLC Waters Symmetry C18, 250 X 4.6 mm column. The gradient runs from 100% solvent A (methanol, water, phosphoric acid (15/85/0.001M, v/v/M)) to 100% solvent B (methanol, water, phosphoric acid (85/15/0.001 M, v/v/M)) in 10 min with a hold at 100% B of 10 min. The flow rate is set at 1 ml/min. The chromatography is carried out at 40° C.

Mass spectrometric measurements in LC/MS mode are performed as follows:

HPLC Conditions

Analyses are performed using a WATERS Alliance HPLC system mounted with an INERTSIL ODS 3, DP 5 μm, 250×4.6 mm column.

The gradient runs from 100% solvent A (acetonitrile, water, trifluoroacetic acid (10/90/0.1, v/v/v)) to 100% solvent B (acetonitrile, water, trifluoroacetic acid (90/10/0.1, v/v/v)) in 7 min with a hold at 100% B of 4 min. The flow rate is set at 2.5 ml/min and a split of 1/25 is used just before API source.

MS Conditions

Samples are dissolved in acetonitrile/water, 70/30, v/v at the concentration of about 250 μg/ml. API spectra (+ or −) are performed using a FINNIGAN LCQ ion trap mass spectrometer. APCI source operated at 450° C. and the capillary heater at 160° C. ESI source operated at 3.5 kV and the capillary heater at 210° C.

Mass spectrometric measurements in DIP/EI mode are performed as follows: samples are vaporized by heating the probe from 50° C. to 250° C. in 5 min. EI (Electron Impact) spectra are recorded using a FINNIGAN TSQ 700 tandem quadrupole mass spectrometer. The source temperature is set at 150° C.

Mass spectrometric measurements on a TSQ 700 tandem quadrupole mass spectrometer (Finnigan MAT) in GC/MS mode are performed with a gas chromatograph model 3400 (Varian) fitted with a split/splitless injector and a DB-5MS fused-silica column (15 m×0.25 mm I.D., 1 μm) from J&W Scientific. Helium (purity 99.999%)/0) is used as carrier gas. The injector (CTC A200S autosampler) and the transfer line operate at 290 and 250° C., respectively. Sample (1 μl) is injected in splitless mode and the oven temperature is programmed as follows: 50° C. for 5 min., increasing to 280° C. (23° C./min) and holding for 10 min. The TSQ 700 spectrometer operates in electron impact (EI) or chemical ionization (Cl/CH₄) mode (mass range 33-800, scan time 1.00 sec). The source temperature is set at 150° C.

Specific rotation is recorded on a Perkin-Elmer 341 polarimeter. The angle of rotation is recorded at 25° C. on 1% solutions in methanol, at 589 nm.

Melting points are determined on a Büchi 535 or 545 Tottoli-type fusionometre, and are not corrected, or by the onset temperature on a Perkin Elmer DSC 7.

Preparative chromatographic separations are performed on silicagel 60 Merck, particle size 15-40 μm, reference 1.15111.9025, using Novasep axial compression columns (80 mm i.d.), flow rates between 70 and 150 ml/min. Amount of silicagel and solvent mixtures as described in individual procedures.

Preparative Chiral Chromatographic separations are performed on a DAICEL Chiralpak AD 20 μm, 100*500 mm column using an in-house build instrument with various mixtures of lower alcohols and C5 to C8 linear, branched or cyclic alkanes at ±350 ml/min. Solvent mixtures as described in individual procedures.

### EXPERIMENTAL

### Example 1

### Synthesis of (5S)-1-{4-[(trans-3-piperidin-1-ylcyclobutyl)oxy]-phenyl}-5-(piperidin-1-ylmethyl)pyrrolidin-2-one 1

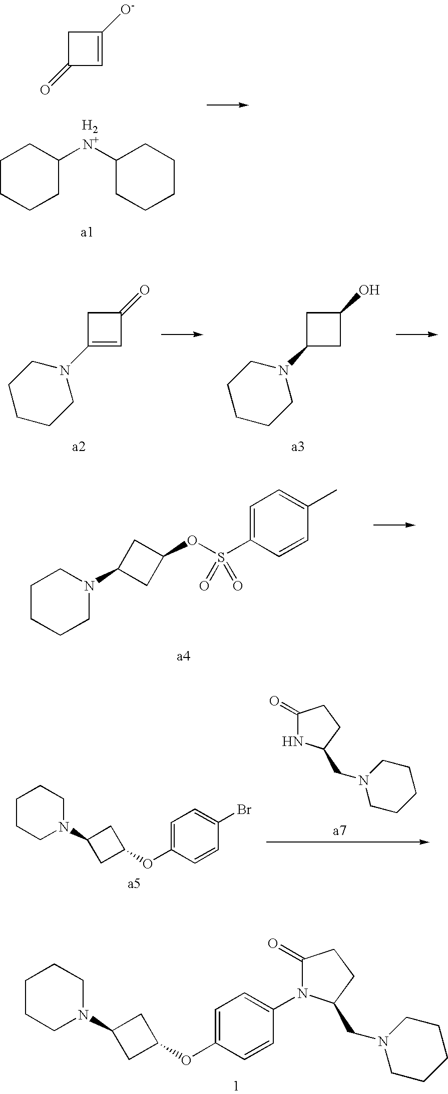

### 1.1 Synthesis of 3-piperidin-1-ylcyclobut-2-en-1-one a2

Trifluoroacetic acid (64 ml, 0.825 mol, 1.1 eq) is added over 10 minutes to a stirred suspension of N-cyclohexylcyclohexanaminium 3-oxocyclobut-1-en-1-olate a1 (200 g, 0.75 mol, 1 eq) in dioxane (1 l). After 4 hours stirring at room temperature, the resulting suspension is filtered and washed with dioxane (300 ml). The filtrate is then stirred at room temperature and treated dropwise with piperidine (96 ml, 0.975 mol, 1.3 eq) while maintaining the temperature below 30° C. throughout the addition (20 minutes) with a water bath. The mixture is stirred overnight at room temperature. The dioxane is then removed under reduced pressure and the resulting oil is taken up in dichloromethane (400 ml). The organic layer is washed with a 1 N aqueous hydrochloric acid solution (400 ml), water (400 ml), a saturated aqueous solution of sodium hydrogen carbonate (400 ml) and brine (400 ml). The organic layer is dried over magnesium sulfate and concentrated to yield 90.7 g of a red solid that is purified by chromatography over silicagel (eluent: dichloromethane/methanol/ammonia 98:1.8:0.2) to afford 74.8 g of 3-piperidin-1-ylcyclobut-2-en-1-one a2.

Yield: 66%.

¹H NMR δ (CDCl₃): 4.47 (s, 1H), 3.22 (m, 4H), 2.95 (s, 2H), 1.53 (m, 6H).

The following compounds may be synthesized according to the same method:

| | | |
|---|---|---|
| a16 | 3-(2-methylpyrrolidin-1-yl)cyclobut-2-en-1-one | ¹H NMR δ (CDCl₃): 4.55 (2 s, J = 6.8 Hz, |
| | | 1 H), 3.94 & 3.81 (2 |
| | | m, 1 H), 3.53 (m, 1 H), 3.32 (m, |
| | | 1 H), 3.16 (m, 1 H), 2.10 (m, 3 |
| | | H), 1.92 (m, 1 H), 1.74 (m, 1 H), |
| | | 1.26 (2 d, J = 6.6 Hz, 3 H). |
| a17 | 3-morpholin-4-ylcyclobut-2-en-1-one | LC-MS (MH⁺): 154 |
| a18 | 3-(4-isopropylpiperazin-1-yl)cyclobut-2-en-1-one | LC-MS (MH⁺): 195 |
| a19 | 3-(4,4-difluoropiperidin-1-yl)cyclobut-2-en-1-one | LC-MS (MH⁺): 188 |
| a20 | 3-pyrrolidin-1-ylcyclobut-2-en-1-one | LC-MS (MH⁺): 138 |
| a21 | 3-azepan-1-ylcyclobut-2-en-1-one | LC-MS (MH⁺): 166 |
| a22 | 3-[(3R)-3-(dimethylamino)pyrrolidin-1-yl]cyclobut- | LC-MS (MH⁺): 181 |
| | 2-en-1-one | |
| a23 | 3-thiomorpholin-4-ylcyclobut-2-en-1-one | LC-MS (MH⁺): 170 |

### 1.2 Synthesis of cis-3-piperidin-1-ylcyclobutanol a3

A solution of 3-piperidin-1-ylcyclobut-2-en-1-one a2 (10 g, 66.1 mmol, 1 eq) in ethanol (200 ml) is treated with portions of sodium borohydride (8.76 g, 231 mmol, 3.5 eq). At the end of the addition, the mixture is stirred at 50° C. for 12 h, cooled down to 20° C. and treated with acetone (20 ml). The solvents are removed under reduced pressure to leave a yellow oil which is diluted with ethyl acetate (200 ml). This organic layer is washed with a saturated aqueous solution of sodium hydrogen carbonate (100 ml), water (100 ml) and brine (100 ml), then concentrated under reduced pressure. The residual oil is purified by chromatography over silicagel (eluent: dichloromethane/methanol/ammonia 95:4.5:0.5) to afford 8 g of cis-3-piperidin-1-ylcyclobutanol a3 as a white solid.

Yield: 78%.

¹H NMR δ (CDCl₃): 3.81 (m, 3H), 2.38 (m, 2H), 2.06 (m, 4H), 1.69 (m, 2H), 1.43 (m, 4 H), 1.29 (bs, 2H).

| | | |
|---|---|---|
| a24 | cis-3-(2-methylpyrrolidin-1-yl)cyclobutanol | ¹H NMR δ (CDCl₃): 3.98 (m, 1 |
| | | H), 2.97 (m, 1 H), 2.48 (m, 4 H), |
| | | 2.20 (q, J = 8.8 Hz, 1 H), |
| | | 1.93 (m, 3 H), 1.71 (m, 3 H), 1.43 (m, |
| | | 1 H), 1.08 (d, J = 6.2 Hz, 3 H). |
| a25 | cis-3-morpholin-4-ylcyclobutanol | LC-MS (MH⁺): 158 |
| a26 | cis-3-(4-isopropylpiperazin-1-yl)cyclobutanol | LC-MS (MH⁺): 199 |
| a27 | cis-3-(4,4-difluoropiperidin-1-yl)cyclobutanol | LC-MS (MH⁺): 192 |
| a28 | cis-3-pyrrolidin-1-ylcyclobutanol | LC-MS (MH⁺): 142 |
| a29 | cis-3-azepan-1-ylcyclobutanol | LC-MS (MH⁺): 170 |
| a30 | cis-3-[(3R)-3-(dimethylamino)pyrrolidin-1- | LC-MS (MH⁺): 185 |
| | yl]cyclobutanol | |
| a31 | cis-3-thiomorpholin-4-ylcyclobutanol | LC-MS (MH⁺): 174 |

### 1.3 Synthesis of cis-3-piperidin-1-ylcyclobutyl 4-methylbenzenesulfonate a4

A solution of cis-3-piperidin-1-ylcyclobutanol a3 (1.0 g, 6.44 mmol, 1.0 eq) and N-methylimidazole (1.03 ml, 12.88 mmol, 2.0 eq) in dichloromethane (10 ml) is treated with p-toluenesulfonyl chloride (2.1 g, 10.95 mmol, 1.7 eq). The mixture is stirred at 20° C. for 48 h. The resulting mixture is washed with a saturated aqueous solution of sodium hydrogen carbonate (10 ml), dried over magnesium sulfate and concentrated to afford 1.8 g of a red oil. This oil is purified by chromatography over silicagel (eluent: dichloromethane/methanol/ammonia 99:0.9:0.1) to yield 1.1 g of cis-3-piperidin-1-ylcyclobutyl 4-methylbenzenesulfonate a4 as an orange solid.

Yield: 55%.

LC-MS (MH⁺): 310.

| | | |
|---|---|---|
| a32 | cis-3-(2-methylpyrrolidin-1-yl)cyclobutyl 4- | LC-MS (MH⁺): 310 |
| | methylbenzenesulfonate | |
| a33 | cis-3-(2-methylpyrrolidin-1-yl)cyclobutyl 4- | |
| | bromobenzenesulfonate | |
| a34 | cis-3-morpholin-4-ylcyclobutyl 4-methylbenzenesulfonate | LC-MS (MH⁺): 312 |
| a35 | cis-3-(4-isopropylpiperazin-1-yl)cyclobutyl 4- | LC-MS (MH⁺): 353 |
| | methylbenzenesulfonate | |
| a36 | cis-3-(4,4-difluoropiperidin-1-yl)cyclobutyl 4- | LC-MS (MH⁺): 346 |
| | methylbenzenesulfonate | |
| a37 | cis-3-pyrrolidin-1-ylcyclobutyl 4-methylbenzenesulfonate | LC-MS (MH⁺): 296 |
| a38 | cis-3-azepan-1-ylcyclobutyl 4-methylbenzenesulfonate | LC-MS (MH⁺): 324 |
| a39 | cis-3-[(3R)-3-(dimethylamino)pyrrolidin-1-yl]cyclobutyl 4- | LC-MS (MH⁺): 339 |
| | methylbenzenesulfonate | |
| a40 | cis-3-thiomorpholin-4-ylcyclobutyl 4-methylbenzenesulfonate | LC-MS (MH⁺): 328 |

### 1.4 Synthesis of compounds of formula II

### 1.4.1 Synthesis of 1-[trans-3-(4-bromophenoxy)cyclobutyl]piperidine a5.

A solution of 4-bromophenol (0.45 g, 2.58 mmol, 1 eq) in dry N,N-dimethylformamide (15 ml) is treated with sodium hydride (60% dispersion in mineral oil, 0.21 g, 5.16 mmol, 2 eq) under an argon atmosphere. After 15 minutes, cis-3-piperidin-1-ylcyclobutyl 4-methylbenzenesulfonate a4 (0.80 g, 2.58 mmol, 1 eq) is added and the mixture is stirred at 80° C. overnight. The mixture is concentrated under reduced pressure, diluted with ethyl acetate (20 ml) and washed twice with a saturated aqueous solution of sodium hydrogen carbonate. The organic layer is then dried over magnesium sulfate and concentrated under reduced pressure. The residue is purified by chromatography over silicagel (eluent: dichloromethane/ethanol 98:2) to afford 1-[trans-3-(4-bromophenoxy)cyclobutyl]piperidine a5 as an orange oil (0.415 g).

Yield: 52%.

LC-MS (MH⁺): 310/312.

1-[trans-3-(3-bromophenoxy)cyclobutyl]piperidine a41 (LC-MS (MH⁺): 310/312) may be synthesized according to the same method.

### 1.4.2 Synthesis of 1-[trans-3-(4-iodophenoxy)cyclobutyl]piperidine a42.

A solution of 4-iodophenol (15.4 g, 70.3 mmol, 1.5 eq) in dry N,N-dimethylformamide (65 ml) is treated with sodium hydride (60% dispersion in mineral oil, 2.0 g, 84.3 mmol, 1.8 eq) under an argon atmosphere. After 30 minutes, cis-3-piperidin-1-ylcyclobutyl 4-methylbenzenesulfonate a4 (14.5 g, 46.9 mmol, 1 eq) is added and the mixture is stirred at 70° C. for 2 days. The mixture is diluted with ethyl acetate and washed with brine. The organic layer is then dried over magnesium sulfate and concentrated under reduced pressure. The residue is purified by chromatography over silicagel (gradient: from dichloromethane 100% to dichloromethane/ethanol/ammonia 97:2.7:0.3) to afford 1-[trans-3-(4-iodophenoxy)cyclobutyl]piperidine a42 as an orange solid (11.5 g).

Yield: 69%.

LC-MS (MH⁺): 358.

1-[trans-3-(4-iodophenoxy)cyclobutyl]-2-methylpyrrolidine a43 (LC-MS (MH⁺): 358) may be synthesized according to the same method. Its enantiomers may be separated by chiral chromatography (Chiralcel O J-H column; isopropanol/benzenz/diethylamine 10/90/0.1)

| | | |
|---|---|---|
| a43 | 1-[trans-3-(4-iodophenoxy)cyclobutyl]-2- | LC-MS (MH⁺): 358 |
| | methylpyrrolidine | |
| a44 | 1-[trans-3-(4-iodophenoxy)cyclobutyl]- | LC-MS (MH⁺): 358 |
| | 2-methylpyrrolidine enantiomer 1 | |
| a45 | 1-[trans-3-(4-iodophenoxy)cyclobutyl]- | LC-MS (MH⁺): 358 |
| | 2-methylpyrrolidine enantiomer 2 | |

### 1.5 Synthesis of pyrrolidin-2-one derivatives

### 1.5.1 Synthesis of (5S)-5-(piperidin-1-ylmethyl)pyrrolidin-2-one a7.

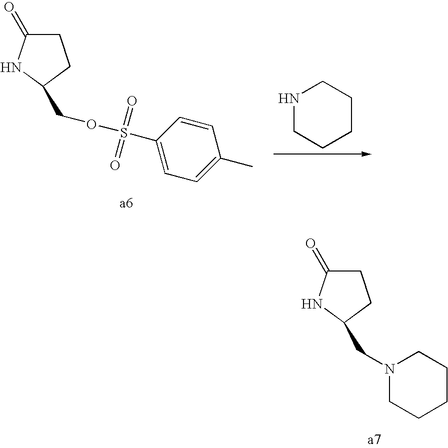

Piperidine (0.7 g, 8.3 mmol, 1.5 eq) is added to a suspension of [(2S)-5-oxopyrrolidin-2-yl]methyl 4-methylbenzenesulfonate a6 (1.5 g, 5.56 mmol, 1 eq) and potassium carbonate (1.5 g, 11.1 mmol, 2 eq) in acetonitrile (50 ml), and the mixture is stirred at reflux overnight. Potassium carbonate is filtered and the solvent is removed under vacuum. The residue is dissolved in a minimum of dichloromethane, the organic layer is sonicated and heated to precipitate a white solid which is filtered. The filtrate is concentrated under vacuum to give 1 g of (5S)-5-(piperidin-1-ylmethyl)pyrrolidin-2-one a7 as a yellow oil.

Yield: 100%.

LC-MS (MH⁺): 183.

The following compounds may be synthesized according to the same method:

| | | |
|---|---|---|
| a8 | (5S)-5-(pyrrolidin-1-ylmethyl)pyrrolidin- | LC-MS (MH⁺): 169 |
| | 2-one | |
| a9 | (5S)-5-(morpholin-4-ylmethyl)pyrrolidin- | LC-MS (MH⁺): 185 |
| | 2-one | |
| a10 | (5S)-5-[(4,4-difluoropiperidin-1- | LC-MS (MH⁺): 219 |
| | yl)methyl]pyrrolidin-2-one | |
| a11 | 5-{[(2,2,2- | LC-MS (MH⁺): 205 |
| | trifluoroethyl)amino]methyl}pyrrolidin-2-one | |
| a12 | (5S)-5-[(3,3-difluoropyrrolidin-1- | LC-MS (MH⁺): 197 |
| | yl)methyl]pyrrolidin-2-one | |

### 1.5.2 Synthesis of (5S)-5-(cyclohexylmethyl)morpholin-3-one a15.

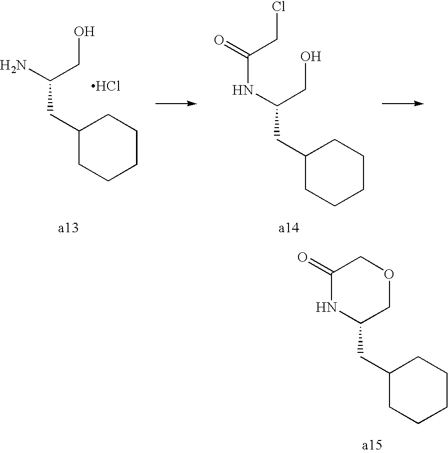

(i) (2S)-2-amino-3-cyclohexylpropan-1-ol hydrochloride a13 (1.25 g, 6.45 mmol, 1.0 eq) is added to a stirred solution of potassium carbonate (2.67 g, 19.35 mmol, 3.0 eq) in a 1:1 mixture of tetrahydrofuran and water (24 ml). The mixture is cooled to 0° C. with an ice bath and chloroacetyl chloride (720 μl, 9.03 mmol, 1.4 eq) is added dropwise. The ice bath is removed and the mixture is stirred at 20° C. for 30 minutes. The mixture is then poured into ethyl acetate (30 ml) and washed with a saturated aqueous solution of sodium hydrogencarbonate. The aqueous layer is back extracted with ethyl acetate (30 ml). The combined organic layers are dried over magnesium sulfate and concentrated to afford 1.53 g of 2-chloro-N-[(1S)-2-cyclohexyl-1-(hydroxymethyl)ethyl]acetamide a14 as a colorless oil which is used in the next step without further purification.

Yield: 100%.

LC-MS (MH⁺): 234/236.

(ii) A solution of 2-chloro-N-[(1S)-2-cyclohexyl-1-(hydroxymethyl)ethyl]acetamide a14 (1.53 g, 6.45 mmol, 1.0 eq) in isopropanol (5 ml) is added dropwise to a stirred suspension of potassium tert-butoxide (1.81 g, 16.12 mmol, 2.5 eq) in isopropanol (10 ml). The mixture is stirred for 1 h at 20° C. and stored at 4° C. overnight (the reaction is complete after 1 h), then treated with 1 N aqueous hydrogen chloride (5.2 ml) and concentrated under reduced pressure. The resulting aqueous suspension is diluted with water (20 ml) and extracted with dichloromethane (2×30 ml). The combined organic layers are dried over magnesium sulfate and concentrated to give 1.05 g of (5S)-5-(cyclohexylmethyl)morpholin-3-one a15 as a white solid.

Yield: 83%.

LC-MS (MH⁺): 198.

(5R)-5-(cyclohexylmethyl)morpholin-3-one a46 may be prepared according to the same method.

LC-MS (MH⁺): 198.

### 1.5.3 Synthesis of (5S)-5-(morpholin-4-ylmethyl)pyrrolidin-2-one a9 and [(2S)-5-oxopyrrolidin-2-yl]methyl morpholine-4-carboxylate a47

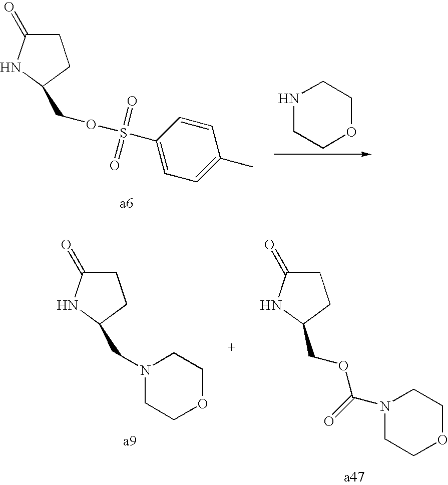

Morpholine (2.43 g, 27.85 mmol, 1.5 eq) is added to a suspension of [(2S)-5-oxopyrrolidin-2-yl]methyl 4-methylbenzenesulfonate a6 (5 g, 18.57 mmol, 1 eq) and potassium carbonate (5.13 g, 37.13 mmol, 2 eq) in acetonitrile (200 ml), and the mixture is stirred at reflux overnight. Potassium carbonate is filtered and the solvent is removed under vacuum. The residue is dissolved in a minimum of dichloromethane, then the organic layer is sonicated and heated to precipitate as a white solid which is filtered. The filtrate is concentrated under vacuum to give 6 g of a mixture of (5S)-5-(morpholin-4-ylmethyl)pyrrolidin-2-one a9 and [(2S)-5-oxopyrrolidin-2-yl]methyl morpholine-4-carboxylate a47.

This mixture is dissolved in a mixture of tetrahydrofuran (50 ml) and 5N aqueous hydrochloric acid (5 ml). A precipitate forms and is filtered. The solid is taken up in dichloromethane and washed with a saturated aqueous solution of sodium hydrogen carbonate. The organic layer is dried over magnesium sulfate and concentrated to yield 3.5 g of (5S)-5-(morpholin-4-ylmethyl)pyrrolidin-2-one a9. The filtrate is taken up in dichloromethane and washed with a saturated aqueous solution of sodium hydrogen carbonate. The organic layer is dried over magnesium sulfate and concentrated to yield 0.2 g of [(2S)-5-oxopyrrolidin-2-yl]methyl morpholine-4-carboxylate a47.

(5S)-5-(morpholin-4-ylmethyl)pyrrolidin-2-one a9:

Yield (crude): 96%.

LC-MS (MH⁺): 185.

[(2S)-5-oxopyrrolidin-2-yl]methyl morpholine-4-carboxylate a47:

Yield (crude): 4%.

LC-MS (MH⁺): 229.

### 1.6 Synthesis of (5S)-1-1-{4-[(3-piperidin-1-ylcyclobutyl)oxy]phenyl}-5-(piperidin-1-ylmethyl)pyrrolidin-2-one 1

A suspension of 1-[trans-3-(4-bromophenoxy)cyclobutyl]piperidine a5 (0.41 g, 1.32 mmol, 1 eq) in dioxane (15 ml), potassium phosphate (0.56 g, 2.64 mmol, 2 eq), copper iodide (5.0 mg, 0.01 mmol, 1 mol %), trans-1,2-diaminocyclohexane (8.0 mg, 0.07 mmol, 10 mol %) and (5S)-5-(piperidin-1-ylmethyl)pyrrolidin-2-one a7 (0.20 g, 1.59 mmol, 1.2 eq) is placed in a sealed tube under argon atmosphere and heated at 110° C. for 3 days. The mixture is diluted with dichloromethane and washed twice with 1 M aqueous sodium hydroxide. The organic layer is dried over magnesium sulfate and concentrated under vacuum to give 279 mg of a brown oil. This oil is purified by chromatography over silicagel (eluent: dichloromethane/ethanol/ammonia 95:5:0.5) to afford (5S)-1-{4-[(3-piperidin-1-ylcyclobutyl)oxy]phenyl}-5-(piperidin-1-ylmethyl)pyrrolidin-2-one 1 (102 mg) as a pale yellow solid.

Yield: 17%. LC-MS (MH⁺): 412.

Compounds 2, 3, 4, 5, 6, 7, 8, 10, 11, 21, 22 and 23 may be synthesized according to the same method.

### Example 2

### Synthesis of (4R)-4-(morpholin-4-ylmethyl)-3-{4-[(trans-3-piperidin-1-ylcyclobutyl)oxy]phenyl}-1,3-oxazolidin-2-one 13

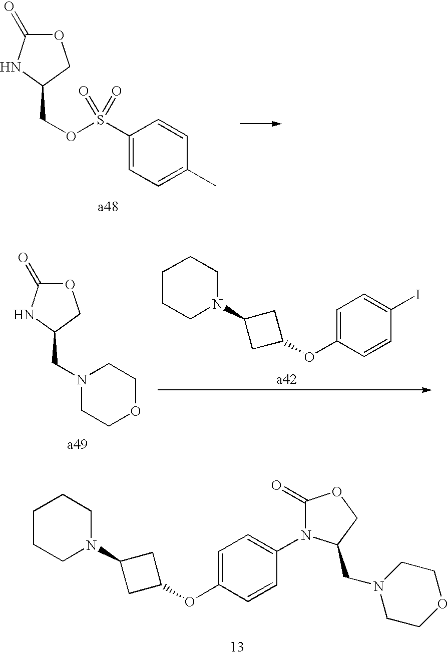

### 2.1 Synthesis of (4R)-4-(morpholin-4-ylmethyl)-1,3-oxazolidin-2-one a49

Potassium carbonate (406 mg, 2.94 mmol, 2 eq) and morpholine (195 μl, 2.21 mmol, 1.5 eq) are added to a solution of [(4S)-2-oxo-1,3-oxazolidin-4-yl]methyl 4-methylbenzenesulfonate a48 (400 mg, 1.47 mmol, 1 eq) in acetonitrile (15 ml) and the mixture is heated at reflux temperature overnight. The mixture is filtered and concentrated under vacuum. The residue is taken up with hot dichloromethane. The remaining undissolved solid is filtered off and the filtrate is concentrated under vacuum. This residue is taken up with a hot mixture of dichloromethane/diethyl ether/hexane and the solid fraction again discarded. The filtrate is concentrated under vacuum to give 251 mg of (4R)-4-(morpholin-4-ylmethyl)-1,3-oxazolidin-2-one a49 as a yellow oil.

Yield: 92%.

LC-MS (MH⁺): 187.

### 2.3 Synthesis of (4R)-4-(morpholin-4-ylmethyl)-3-{4-[(trans-3-piperidin-1-ylcyclobutyl)oxy]phenyl}-1,3-oxazolidin-2-one 13

A suspension of potassium phosphate (573 mg, 2.7 mmol, 2 eq), copper iodide (3 mg, 0.014 mmol, 1 mol %), trans-1,2-diaminocyclohexane (16 mg, 0.14 mmol, 10 mol %), 1-[trans-3-(4-iodophenoxy)cyclobutyl]piperidine a42 (482 mg, 1.35 mmol, 1 eq) and (4R)-4-(morpholin-4-ylmethyl)-1,3-oxazolidin-2-one a49 (251 mg, 1.35 mmol, 1 eq) in dioxane (7 ml) is placed in a sealed tube under an argon atmosphere and heated at 100° C. for 2 days. The mixture is diluted with ethyl acetate and washed twice with a 1 N aqueous solution of sodium hydroxide. The aqueous phase is extracted with ethyl acetate and the combined organic phases are dried over magnesium sulfate and concentrated under vacuum to give 615 mg of brown oil. The oil is purified by chromatography over silicagel (dichloromethane/methanol/ammonia 94:6:0.6) to afford 390 mg of (4R)-4-(morpholin-4-ylmethyl)-3-{4-[(trans-3-piperidin-1-ylcyclobutyl)oxy]phenyl}-1,3-oxazolidin-2-one 13 as a yellow oil.

Yield: 70%.

LC-MS (MH⁺): 416.

Compounds 12 and 15 may be synthesized according to the same method.

### Example 3

### Synthesis of 5-[(4,4-difluoropiperidin-1-yl)methyl]-4-{4-[(trans-3-piperidin-1-ylcyclobutyl)oxy]phenyl}morpholin-3-one 9

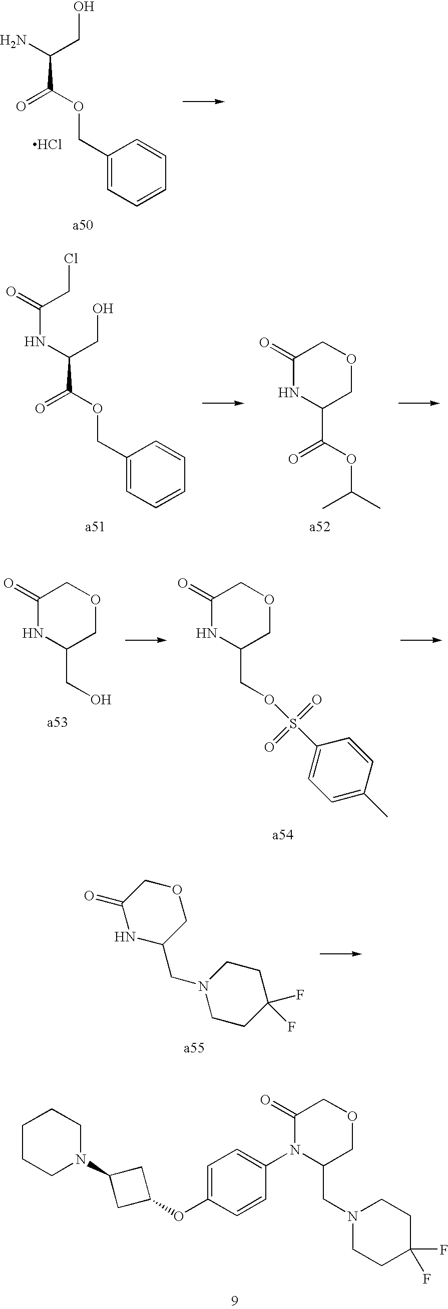

### 3.1 Synthesis of benzyl (2S)-2-[(chloroacetyl)amino]-3-hydroxypropanoate a51

Chloroacetylchloride (2.9 ml, 36 mmol, 1.7 eq) is added dropwise to a solution of potassium carbonate (8.95 g, 65 mmol, 3 eq) and L-serine benzyl ester hydrochloride a50 (5 g, 21 mmol, 1 eq) in a 1:1 tetrahydrofuran-water mixture (80 ml) at 0° C. The mixture is stirred at room temperature for 1 hour, diluted with ethyl acetate and washed with a saturated solution of sodium hydrogenocarbonate. The aqueous phase is extracted with ethyl acetate, the combined organic phases are dried over magnesium sulfate and concentrated under vacuum to afford 5.59 g of benzyl (2S)-2-[(chloroacetyl)amino]-3-hydroxypropanoate a51 as a white solid.

Yield: 98%.

LC-MS (MH⁺): 272/274.

### 3.2 Synthesis of propan-2-yl 5-oxomorpholine-3-carboxylate a52

A solution of benzyl (2S)-2-[(chloroacetyl)amino]-3-hydroxypropanoate a51 (30 ml) is added dropwise to a suspension of potassium tert-butoxide (5.77 g, 50 mmol, 2.5 eq) in isopropanol (32 ml). The mixture is stirred 1 hour at room temperature, then a 5 N aqueous hydrochloric acid solution (17.4 ml) is added at 0° C. and the alcohol is removed under reduce pressure. Water is added and the aqueous phase is extracted twice with ethyl acetate. The combined organic phases are dried over magnesium sulfate and concentrated under vacuum to give 3.33 g of an orange oil. The residue is taken up with hexane and heated. Hexane is removed with a pipette and the residue is dried under high vacuum to give 2.55 g of propan-2-yl 5-oxomorpholine-3-carboxylate a52 as an orange oil.

Yield: 68%.

LC-MS (MH⁺): 188.

### 3.3 Synthesis of 5-(hydroxymethyl)morpholin-3-one a53

A solution of propan-2-yl 5-oxomorpholine-3-carboxylate a52 (1.4 g, 7.48 mmol, 1 eq) in ethanol (15 ml) is treated with portions of sodium borohydride (297 mg, 7.85 mmol, 1.05 eq) at 0° C. At the end of the addition, the mixture is stirred at room temperature for 5 hours and a saturated solution of ammonium chloride (439 mg, 8.22 mmol, 1.1 eq) is added. The mixture is stirred for 30 minutes. The precipitate is filtered off and the filtrate is concentrated under vacuum to afford 1.36 g of 5-(hydroxymethyl)morpholin-3-one a53 as a semi-solid.

LC-MS (MH⁺): 132.

### 3.4 Synthesis of (5-oxomorpholin-3-yl)methyl 4-methylbenzenesulfonate a54

p-Toluenesulfonylchloride (2.2 g, 11.52 mmol, 1.54 eq) is added to a solution of crude 5-(hydroxymethyl)morpholin-3-one a53 (1.36 g, 7.48 mmol theoretical, 1 eq) in pyridine (3 ml) at 0° C. Dichloromethane is added obtain a clear solution and the mixture is stirred at room temperature overnight. The solvents are removed under reduced pressure. The residue is taken up in dichloromethane and washed twice with a 1 N aqueous solution of hydrochloric acid. The aqueous phase is extracted with dichloromethane and the combined organic phases are dried over magnesium sulfate and concentrated under vacuum to give 563 mg of a brown oil. This oil is taken up with dichloromethane and hexane is added. The precipitate is filtered and dried to afford 300 mg of (5-oxomorpholin-3-yl)methyl 4-methylbenzenesulfonate a54 as a brown solid.

Yield: 14% (over 2 steps).

LC-MS (MH⁺): 286.

### 3.5 Synthesis of 5-[(4,4-difluoropiperidin-1-yl)methyl]morpholin-3-one a55

Potassium carbonate (363 mg, 2.62 mmol, 2,5 eq) and 4,4-difluoropiperidine hydrochloride (247 mg, 1.58 mmol, 1.5 eq) are added to a solution of (5-oxomorpholin-3-yl)methyl 4-methylbenzenesulfonate a54 (300 mg, 1.05 mmol, 1 eq) in acetonitrile (10 ml) and the mixture is heated under reflux overnight. The mixture is filtered and concentrated under vacuum. The residue is taken up with dichloromethane, heated, filtered and concentrated under vacuum to afford 180 mg of 5-[(4,4-difluoropiperidin-1-yl)methyl]morpholin-3-one a55 as a brown oil.

Yield: 73%.

LC-MS (MH⁺): 235.

### 3.6 Synthesis of 5-[(4,4-difluoropiperidin-1-yl)methyl]-4-{4-[(trans-3-piperidin-1-ylcyclobutyl)oxy]phenyl}morpholin-3-one 9

A suspension of potassium phosphate (327 mg, 1.54 mmol, 2 eq), copper iodide (1 mg, 0.008 mmol, 1 mol %), trans-1,2-diaminocyclohexane (9 mg, 0.08 mmol, 10 mol %), 1-[trans-3-(4-iodophenoxy)cyclobutyl]piperidine a42 (274 mg, 0.77 mmol, 1 eq) and 5-[(4,4-difluoropiperidin-1-yl)methyl]morpholin-3-one a55 (180 mg, 0.77 mmol, 1 eq) in dioxane (4 ml) is placed in a sealed tube under argon atmosphere and heated at 100° C. for 2 days. The mixture is diluted with ethyl acetate and washed twice with a 1 N aqueous solution of sodium hydroxide. The aqueous phase is extracted with ethyl acetate and the combined organic phases are dried over magnesium sulfate and concentrated under vacuum to give 412 mg of a brown oil. The oil is purified by chromatography over silicagel (dichloromethane/methanol/ammonia 96:4:0.4 then 95:5:0.5) and then by reverse phase chromatography (acetonitrile/water/trifluoroacetic acid 5:95:0.1) to afford 140 mg of 5-[(4,4-difluoropiperidin-1-yl)methyl]-4-{-4-[(trans-3-piperidin-1-ylcyclobutyl)oxy]phenyl}morpholin-3-one 9 as a trifluoroacetate salt and a colourless lacquer. This salt is taken up with a 0.5 N aqueous solution of sodium hydroxide and extracted three times with dichloromethane. The combined organic phases are dried over magnesium sulfate and evaporated under vacuum to afford 50 mg of 5-[(4,4-difluoropiperidin-1-yl)methyl]-4-{4-[(trans-3-piperidin-1-ylcyclobutyl)oxy]phenyl}morpholin-3-one 9 as a white solid.

Yield: 14%.

LC-MS (MH⁺): 464.

### Example 4

### Synthesis of (4R)-4-(cyclohexylmethyl)-3-{4-[(trans-3-piperidin-1-ylcyclobutyl)oxy]phenyl}-1,3-oxazolidin-2-one 14

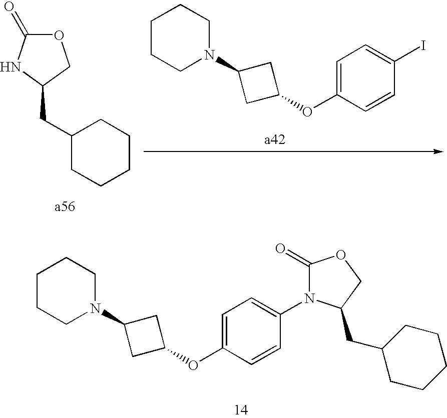

A suspension of potassium phosphate (753 mg, 3.55 mmol, 2 eq), copper iodide (4 mg, 0.018 mmol, 1 mol %), trans-1,2-diaminocyclohexane (20 mg, 0.18 mmol, 10 mol %), 1-[trans-3-(4-iodophenoxy)cyclobutyl]piperidine a42 (634 mg, 1.77 mmol, 1 eq) and (4R)-4-(cyclohexylmethyl)-1,3-oxazolidin-2-one a56 (325 mg, 1.77 mmol, 1 eq) in dioxane (10 ml) is placed in a sealed tube under argon atmosphere and heated at 110° C. for 6 days. The mixture is diluted with ethyl acetate and washed twice with a 1 N aqueous solution of sodium hydroxide. The aqueous phase is extracted with ethyl acetate and the combined organic phases are dried over magnesium sulfate and concentrated under vacuum to give 842 mg of brown oil. The oil is purified by chromatography over silicagel (dichloromethane/methanol/ammonia 94:6:0.6) to afford 180 mg of (4R)-4-(cyclohexylmethyl)-3-{4-[(trans-3-piperidin-1-ylcyclobutyl)oxy]phenyl}-1,3-oxazolidin-2-one

14 as an orange lacquer.

Yield: 29%.

LC-MS (MH⁺): 413.

### Example 5

### Synthesis of (5S)-5-(morpholin-1-ylmethyl)-1-{4-[(3-piperidin-1-ylcyclobutyl)thio]phenyl}pyrrolidin-2-one 16

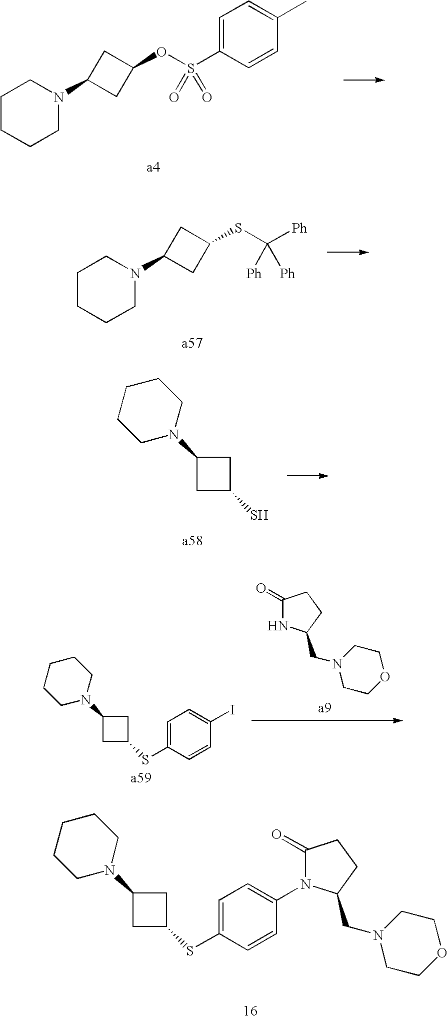

### 5.1 Synthesis of 1-[trans-3-(tritylsulfanyl)cyclobutyl]piperidine a57

A solution of triphenylmethanethiol (4.60 g, 16.64 mmol, 1.3 eq) in dry N,N-dimethylformamide (20 ml) is treated with sodium hydride (60% dispersion in mineral oil, 620 mg, 15.50 mmol, 1.2 eq) under an argon atmosphere. After 15 minutes, a solution of cis-3-piperidin-1-ylcyclobutyl 4-methylbenzenesulfonate a4 (4.0 g, 12.93 mmol, 1 eq) in N,N-dimethylformamide (15 ml) is added and the mixture is stirred at 50° C. overnight. Water is added and the mixture is extracted with dichloromethane. The organic layer is dried over magnesium sulfate and concentrated under reduced pressure. The residue is purified by chromatography over silicagel (dichloromethane/methanol/ammonia 98.5:1.35:0.15) to afford 4.25 g of 1-[trans-3-(tritylsulfanyl)cyclobutyl]piperidine a57 as a brown oil.

Yield: 79%.

LC-MS (MH⁺): 414.

### 5.2 Synthesis of trans-3-[butyl(ethyl)amino]cyclobutanethiol a58

Triethylsilane (1.8 ml, 11.27 mmol, 1.1 eq) and trifluoroacetic acid (20 ml, 269 mmol, 26 eq) are successively added at 5° C. to a solution of 1-[trans-3-(tritylsulfanyl)cyclobutyl]piperidine a57 (4.25 g, 10.27 mmol, 1 eq) in dichloromethane (20 ml). The reaction mixture is stirred at room temperature for 1 h30, then concentrated under reduced pressure. The residue is diluted with ethyl acetate, washed with a 10% aqueous sodium hydrogen carbonate solution and extracted thrice with ethyl acetate. The organic layer is dried over magnesium sulfate and concentrated under reduced pressure. The crude is purified by chromatography over silicagel (dichloromethane/methanol/ammonia 97:2.7:0.3) to afford 800 mg of trans-3-[butyl(ethyl)amino]cyclobutanethiol a58 as an orange oil.

Yield: 45%.

¹H NMR (CDCl₃) δ 3.50 (m, 1H), 3.09 (quint, J=7.3 Hz, 1H), 2.50 (m, 1H), 2.24 (m, 4 H), 2.06 (m, 2H), 1.86 (d, J=6.1 Hz, 2H), 1.58 (m, 4H), 1.45 (m, 2H).

### 5.3 Synthesis of 1-{trans-3-[(4-iodophenyl)sulfanyl]cyclobutyl}piperidine a59

A solution of trans-3-[butyl(ethyl)amino]cyclobutanethiol a58 (774 mg, 4.52 mmol, 1 eq) in dry N,N-dimethylformamide (14 ml) is treated with sodium hydride (60% dispersion in mineral oil, 200 g, 5.0 mmol, 1.1 eq) under an argon atmosphere. After 15 minutes, 4-fluoro-1-iodobenzene (1.0 ml, 8.67 mmol, 1.9 eq) is added and the mixture is stirred at 60° C. for 3 hours. Water is added and the mixture is extracted with dichloromethane. The organic layer is then dried over magnesium sulfate and concentrated under reduced pressure. The crude is purified by chromatography over silicagel (dichloromethane/methanol/ammonia 98:2:0.2) to afford 680 mg of 1-{trans-3-[(4-iodophenyl)sulfanyl]cyclobutyl}piperidine a59 as an beige solid.

Yield: 40%.

¹H NMR (DMSO) δ 7.63 (d, J=8.4 Hz, 2H), 6.98 (d, J=8.4 Hz, 2H), 3.83 (m, 1H), 2.94 (m, 1H), 2.39 (m, 2H), 2.17 (m, 4H), 1.96 (m, 2H), 1.47 (m, 4H), 1.35 (m, 2H).

### 5.4 Synthesis (5S)-5-(morpholin-4-ylmethyl)-1-{4-[(trans-3-piperidin-1-ylcyclobutyl)thio]phenyl}pyrrolidin-2-one 16

A suspension of 1-{trans-3-[(4-iodophenyl)sulfanyl]cyclobutyl}piperidine a59 (300 mg, 0.80 mmol, 1 eq) in dioxane (15 ml), potassium phosphate (761 mg, 3.60 mmol, 4.5 eq), copper iodide (29 mg, 0.15 mmol, 1.9 mol %), trans-1,2-diaminocyclohexane (20 μL, 0.17 mmol, 2.1 mol %) and (5S)-5-(morpholin-4-ylmethyl)pyrrolidin-2-one a9 (327 mg, 1.77 mmol, 2 eq) is placed in a sealed tube under argon atmosphere and heated overnight at 100° C. The mixture is diluted with dichloromethane, washed with a 1 M aqueous sodium hydroxide solution and extracted thrice with dichloromethane. The organic layer is dried over magnesium sulfate and concentrated under vacuum to give 600 mg of a brown oil. This oil is purified by chromatography on silicagel (dichloromethane/methanol 96:4 to 94:6) to afford 280 mg of (5S)-5-(morpholin-4-ylmethyl)-1-{4-[(trans-3-piperidin-1-ylcyclobutyl)thio]phenyl}pyrrolidin-2-one 16 as a beige solid.

Yield: 81%.

LC-MS (MH⁺): 430.

(5S)-5-[(4,4-difluoropiperidin-1-yl)methyl]-1-{4-[(trans-3-piperidin-1-ylcyclobutyl)thio]phenyl}pyrrolidin-2-one 17 may be synthesized according to the same method.

### Example 6

### Synthesis of (4S)-4-(morpholin-4-ylcarbonyl)-3-{4-[(trans-3-piperidin-1-ylcyclobutyl)oxy]phenyl}-1,3-oxazolidin-2-one 18

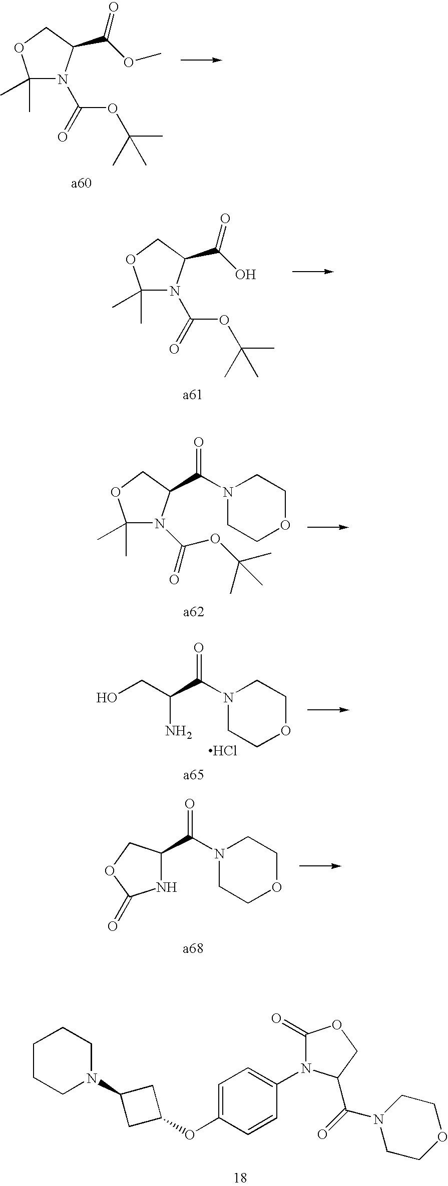

### 6.1. Synthesis of (4S)-3-(tert-butoxycarbonyl)-2,2-dimethyl-1,3-oxazolidine-4-carboxylic acid a61

Lithium hydroxide (277 mg, 11.5 mmol, 1 eq) is added to a solution of methyl 3-tert-butyl 4-methyl (4S)-2,2-dimethyl-1,3-oxazolidine-3,4-dicarboxylate a60 (3 g, 11.5 mmol, 1 eq) in a tetrahydrofuran/water mixture (23 ml/11 ml). The mixture is stirred at room temperature for 2 days, acidified to pH 4 with a 1 N aqueous solution of hydrochloric acid and extracted three times with ethyl acetate. The combined organic phases are dried over magnesium sulfate and concentrated under vacuum to afford 2.79 g of (4S)-3-(tert-butoxycarbonyl)-2,2-dimethyl-1,3-oxazolidine-4-carboxylic acid a61 as a yellow oil.

Yield: 99%.

LC-MS (MH⁺): 246.

### 6.2 Synthesis of tert-butyl (4S)-2,2-dimethyl-4-(morpholin-4-ylcarbonyl)-1,3-oxazolidine-3-carboxylate a62

1-(3-Dimethylaminopropyl)-3-ethylcarbodiimide hydrochloride (740 mg, 3.86 mmol, 1.1 eq) and 1-hydroxybenzotriazole hydrate (522 mg, 3.86 mmol, 1.1 eq) are added to a cold solution (0° C.) of (4S)-3-(tert-butoxycarbonyl)-2,2-dimethyl-1,3-oxazolidine-4-carboxylic acid a61 (860 mg, 3.50 mmol, 1 eq) and morpholine (336 μl, 3.86 mmol, 1.1 eq) in N,N-dimethylformamide (30 ml). The mixture is stirred at room temperature for 6 hours and concentrated to dryness. The residue is dissolved in a 0.5 N aqueous solution of hydrochloric acid and extracted three times with dichloromethane. The combined organic phases are washed with a saturated solution of sodium hydrogenocarbonate, dried over magnesium sulfate and concentrated under vacuum to afford 991 mg of tert-butyl (4S)-2,2-dimethyl-4-(morpholin-4-ylcarbonyl)-1,3-oxazolidine-3-carboxylate a62 as a yellow solid.

Yield: 90%.

LC-MS (MH⁺): 315.

The following compounds may be synthesized according to the same method:

| | | |
|---|---|---|
| a63 | tert-butyl (4S)-2,2-dimethyl-4-(piperidin- | LC-MS (MH⁺): 313 |
| | 1-ylcarbonyl)-1,3- | |
| | oxazolidine-3-carboxylate | |
| a64 | tert-butyl (4S)-4-[(4,4-difluoropiperidin- | LC-MS (MH⁺): 349 |
| | 1-yl)carbonyl]-2,2- | |
| | dimethyl-1,3-oxazolidine-3-carboxylate | |

### 6.3 Synthesis of (2S)-2-amino-3-hydroxy-1-(morpholin-4-yl)propan-1-one hydrochloride a65

Trifluoroacetic acid (1.97 ml, 26.5 mmol, 10 eq) is added to a solution of tert-butyl (4S)-2,2-dimethyl-4-(morpholin-4-ylcarbonyl)-1,3-oxazolidine-3-carboxylate a62 (833 mg, 2.65 mmol, 1 eq) in dichloromethane at 0° C. The mixture is stirred at room temperature overnight and concentrated to dryness. The residue is dissolved in 5 N aqueous hydrochloric acid and the mixture is heated at 65° C. overnight. The mixture is concentrated under vacuum to afford 444 mg of (2S)-2-amino-3-hydroxy-1-(morpholin-4-yl)propan-1-one hydrochloride a65.

Yield: 79%.

LC-MS (MH⁺): 175.

The following compounds may be synthesized according to the same method:

| | | |
|---|---|---|
| a66 | (2S)-2-amino-3-hydroxy-1-(piperidin-1- | LC-MS (MH⁺): 173 |
| | yl)propan-1-one hydrochloride | |
| a67 | (2S)-2-amino-1-(4,4-difluoropiperidin-1-yl)- | LC-MS (MH⁺): 209 |
| | 3-hydroxypropan-1-one hydrochloride | |

### 6.4 Synthesis of (4S)-4-(morpholin-4-ylcarbonyl)-1,3-oxazolidin-2-one a68

Triphosgene (311 mg, 1.05 mmol, 0.5 eq) is added to a solution of (2S)-2-amino-3-hydroxy-1-(morpholin-4-yl)propan-1-one hydrochloride a65 (444 mg, 2.1 mmol, 1 eq) and diisopropylethylamine (1.56 ml, 8.96 mmol, 4.25 eq) in dichloromethane (20 ml) at 0° C. and the mixture is stirred overnight. Water (0.5 ml) is added and the mixture is stirred for 1 hour and filtered over silica gel and magnesium sulfate (dichloromethane 100% then dichloromethane/methanol/ammonia 90:10:1) to afford 1.64 g of crude (4S)-4-(morpholin-4-ylcarbonyl)-1,3-oxazolidin-2-one a68 as a yellow solid containing also diisopropylethylamine salts. This mixture is carried through in the next step.

LC-MS (MH⁺): 201.

The following compounds may be synthesized according to the same method:

| | | |
|---|---|---|
| a69 | (4S)-4-(piperidin-1-ylcarbonyl)-1,3- | LC-MS (MH⁺): 199 |
| | oxazolidin-2-one | |
| a70 | (4S)-4-[(4,4-difluoropiperidin-1- | LC-MS (MH⁺): 235 |
| | yl)carbonyl]-1,3-oxazolidin-2-one | |

### 6.5 Synthesis of (4S)-4-(morpholin-4-ylcarbonyl)-3-{4-[(trans-3-piperidin-1-ylcyclobutyl)oxy]phenyl}-1,3-oxazolidin-2-one 18

A suspension of potassium phosphate (2.10 g, 9.9 mmol, 4.7 eq), copper iodide (10 mg, 0.05 mmol, 2 mol %), trans-1,2-diaminocyclohexane (57 mg, 0.5 mmol, 20 mol %), 4-iodo-1-[(3-piperidin-1-ylcyclobutyl)oxy]benzene (1.77 g, 4.95 mmol, 2.3 eq) and crude (4S)-4-(morpholin-4-ylcarbonyl)-1,3-oxazolidin-2-one a68 (1.64 g, 2.1 mmol theoretical, 1 eq) in dioxane (20 ml) is placed in a sealed tube under argon atmosphere and heated at 100° C. for 5 days. The mixture is diluted with ethyl acetate and washed twice with a 1 N aqueous solution of sodium hydroxide. The organic phase is dried over magnesium sulfate and concentrated under vacuum to give 2.21 g of a brown solid. The solid is purified by chromatography over silicagel (dichloromethane/methanol/ammonia 97:3:0.3) to afford 360 mg of (4S)-4-(morpholin-4-ylcarbonyl)-3-{4-[(trans-3-piperidin-1-ylcyclobutyl)oxy]phenyl}-1,3-oxazolidin-2-one 18 as a yellow solid.

Yield: 40% (over 2 steps).

LC-MS (MH⁺): 430.

Compounds 19 and 20 may be synthesized according to the same method.

Table I gives characteristics of some compounds of general formula (I). Said table indicates the stereochemical information in the columns headed “configuration”: the first column indicates whether a compound has no stereogenic center (achiral), is a pure enantiomer (pure), a racemate (rac) or is a mixture of two stereoisomers, possibly in unequal proportions (mixture); the second column contains the stereochemical assignment for the recognized center, following the IUPAC numbering used in the “IUPAC name” column. A number alone indicates the existence of both configurations at that center. A number followed by ‘R’ or ‘S’ indicates the known absolute configuration at that center. A number followed by ‘!’ indicates the existence of only one but unknown absolute configuration at that center. The letter (A, B) in front is a way of distinguishing the various enantiomers of the same structure.

Table I indicates also the IUPAC name of the compound, the ion peak observed in mass spectrometry and the ¹H NMR description and the optical rotation in the case of enantiomerically pure compounds. The expression “enantiomerically pure” as used herein refers to compounds which have an enantiomeric excess (ee) greater than 95%.

TABLE 1
Physical Characterisation of Example Compounds.

| | | | | MH⁺ | | |
|---|---|---|---|---|---|---|
| n^{o} | Configuration | | IUPAC NAME | (M⁺·) | ¹H NMR δ (400 MHz, CDCl₃, ppm) | alpha_{D} |
| 1 | pure | 5S | (5S)-1-{4-[(trans-3-piperidin-1- | 412 | 7.26 (m, 2 H), 6.77 (d, 2 H), 4.72 (m, 1 H), | −17.38 |
| | | trans | ylcyclobutyl)oxy]phenyl}-5-(piperidin- | | 4.21 (m, 1 H), 2.98 (m, 1 H), 2.65 (m, 1 H), | |
| | | | 1-ylmethyl)pyrrolidin-2-one | | 2.46 (m, 2 H), 2.32 (m, 14 H), 2.06 (m, 1 H), | |
| | | | | | 1.61 (m, 4 H), 1.47 (m, 6 H), 1.36 (m, 2 H) | |
| 2 | pure | 5S | (5S)-5-(cyclohexylmethyl)-4-{4- | 427 | 7.10 (d, J = 8.8 Hz, 2 H), 6.80 (d, J = 8.8 Hz, | +13.99 |
| | | trans | [(trans-3-piperidin-1- | | 2 H), 4.74 (m, 1 H), 4.35 (d, J = 16.7 Hz, 1 | |
| | | | ylcyclobutyl)oxy]phenyl}morpholin-3- | | H), 4.25 (d, J = 16.7 Hz, 1 H), 3.98 (dd, J = 11.9, | |
| | | | one | | 2.9 Hz, 1 H), 3.91 (dd, J = 11.9, 2.9 Hz, | |
| | | | | | 1 H), 3.75 (dd, J = 10.3, 3.3 Hz, 1 H), | |
| | | | | | 2.99 (m, 1 H), 2.33 (m, 8 H), 1.61 (m, 10 H), | |
| | | | | | 1.46 (m, 2 H), 1.34 (m, 1 H), 1.12 (m, 4 H), | |
| | | | | | 0.85 (m, 1 H), 0.63 (m, 1 H) | |
| 3 | pure | 5S | (5S)-5-[(4,4-difluoropiperidin-1- | 448 | (DMSO): 7.32 (d, J = 8.8 Hz, 2 H), 6.80 (d, J = 9.0 Hz, | −1.79 |
| | | trans | yl)methyl]-1-{4-[(trans-3-piperidin-1- | | 2 H), 4.73 (m, 1 H), 4.38 (m, 1 H), | |
| | | | ylcyclobutyl)oxy]phenyl}pyrrolidin-2- | | 2.84 (m, 1 H), 2.57 (m, 2 H), 2.45 (m, 1 H), | |
| | | | one bis(trifluoroacetate) | | 2.39 (m, 4 H), 2.31 (m, 4 H), 2.21 (m, 4 H), | |
| | | | | | 2.10 (m, 2 H), 1.88 (m, 1 H), 1.77 (m, 4 H), | |
| | | | | | 1.49 (m, 4 H), 1.38 (m, 2 H) | |
| 4 | pure | 5S | (5S)-5-(morpholin-4-ylmethyl)-1-{4- | 414 | (DMSO): 7.30 (d, J = 9.0 Hz, 2 H), 6.80 (d, | −5.52 |
| | | trans | [(trans-3-piperidin-1- | | J = 9.0 Hz, 2 H), 4.74 (m, 1 H), 4.34 (m, 1 | |
| | | | ylcyclobutyl)oxy]phenyl}pyrrolidin-2- | | H), 3.46 (m, 4 H), 2.85 (m, 1 H), 2.57 (m, 1 | |
| | | | one bis(trifluoroacetate) | | H), 2.34 (m, 7 H), 2.23 (m, 7 H), 2.10 (m, 2 | |
| | | | | | H), 1.92 (m, 1 H), 1.50 (m, 4 H), 1.39 (m, 2 | |
| | | | | | H) | |
| 5 | pure | 5S | (5S)-1-{4-[(trans-3-piperidin-1- | 398 | 7.26 (d, J = 5.0 Hz, 2 H), 6.78 (d, J = 9.0 Hz, | |
| | | trans | ylcyclobutyl)oxy]phenyl}-5-(pyrrolidin- | | 2 H), 4.73 (m, 1 H), 4.21 (m, 1 H), 2.98 (m, 1 | |
| | | | 1-ylmethyl)pyrrolidin-2-one | | H), 2.64 (m, 1 H), 2.53 (m, 2 H), 2.47 (m, 5 | |
| | | | | | H), 2.32 (m, 9 H), 2.10 (m, 1 H), 1.72 (m, 4 | |
| | | | | | H), 1.60 (m, 4 H), 1.46 (m, 2 H) | |
| 6 | pure | 5S | (5S)-1-{4-[(trans-3-piperidin-1- | 426 | 11.56 (m, 1 H), 7.23 (m, 2 H), 6.79 (d, J = 9.0 Hz, | −6.52 |
| | | trans | ylcyclobutyl)oxy]phenyl}-5-{[(2,2,2- | | 2 H), 4.83 (m, 1 H), 4.21 (m, 1 H), | |
| | | | trifluoroethyl)amino]methyl}pyrrolidin- | | 3.64 (m, 3 H), 3.10 (m, 4 H), 2.78 (m, 3 H), | |
| | | | 2-one bis(trifluoroacetate) | | 2.52 (m, 6 H), 2.34 (m, 1 H), 2.08 (m, 1 H), | |
| | | | | | 1.96 (m, 5 H), 1.42 (m, 1 H) | |
| 7 | pure | 5S | (5S)-5-[(3,3-difluoropyrrolidin-1- | 434 | 12.76 (m, 1 H), 7.25 (m, 2 H), 6.77 (d, J = 9.0 Hz, | −8.65 |
| | | trans | yl)methyl]-1-{4-[(trans-3-piperidin-1- | | 2 H), 4.84 (t, J = 6.8 Hz, 1 H), | |
| | | | ylcyclobutyl)oxy]phenyl}pyrrolidin-2- | | 4.20 (m, 1 H), 3.60 (m, 3 H), 3.12 (m, 2 H), | |
| | | | one bis(trifluoroacetate) | | 2.89 (m, 2 H), 2.74 (t, J = 7.0 Hz, 2 H), 2.51 (m, 4 | |
| | | | | | H), 2.21 (m, 2 H), 1.99 (m, 11 H), 1.40 (m, 1 | |
| | | | | | H) | |
| 8 | pure | 5S | (5S)-1-{3-[(trans-3-piperidin-1- | 412 | 7.23 (t, J = 8.5 Hz, 1 H), 7.00 (m, 2 H), | −25.49 |
| | | trans | ylcyclobutyl)oxy]phenyl}-5-(piperidin- | | 6.63 (m, 1 H), 4.75 (m, 1 H), 4.29 (m, 1 H), | |
| | | | 1-ylmethyl)pyrrolidin-2-one | | 3.01 (m, 1 H), 2.68 (m, 1 H), 2.49 (m, 2 H), | |
| | | | | | 2.36 (m, 14 H), 2.08 (m, 1 H), 1.61 (m, 4 H), | |
| | | | | | 1.48 (m, 6 H), 1.37 (m, 2 H) | |
| 9 | rac | trans | 5-[(4,4-difluoropiperidin-1-yl)methyl]- | 464 | 7.12 (m, 2 H), 6.81 (m, 2 H), 4.74 (m, 1 H), | +0.20 |
| | | | 4-{4-[(trans-3-piperidin-1- | | 4.37 (m, 1 H), 4.24 (m, 1 H), 4.21 (d, J = 7.3 Hz, | |
| | | | ylcyclobutyl)oxy]phenyl}morpholin-3- | | 1 H), 3.93 (m, 1 H), 3.64 (m, 1 H), | |
| | | | one | | 2.99 (m, 1 H), 2.79 (m, 1 H), 2.47 (m, 3 H), | |
| | | | | | 2.38 (m, 5 H), 2.31 (m, 5 H), 1.87 (m, 4 H), | |
| | | | | | 1.61 (m, 4 H), 1.46 (m, 2 H) | |
| 10 | pure | 5R | (5R)-5-(cyclohexylmethyl)-4-{4- | 427 | 7.10 (d, J = 8.8 Hz, 2 H), 6.80 (d, J = 8.8 Hz, | −15.33 |
| | | trans | [(trans-3-piperidin-1- | | 2 H), 4.75 (m, 1 H), 4.30 (m, 2 H), 3.94 (m, 2 | |
| | | | ylcyclobutyl)oxy]phenyl}morpholin-3- | | H), 3.74 (m, 1 H), 2.99 (m, 1 H), 2.35 (m, 6 | |
| | | | one | | H), 1.57 (m, 14 H), 1.36 (m, 1 H), 1.12 (m, 4 | |
| | | | | | H), 0.85 (m, 1 H), 0.61 (m, 1 H) | |
| 11 | pure | 2S | [(2S)-5-oxo-1-{4-[(trans-3-piperidin-1- | 458 | 7.32 (d, J = 8.8 Hz, 2 H), 6.86 (m, 2 H), | −9.69 |
| | | trans | ylcyclobutyl)oxy]phenyl}pyrrolidin-2- | | 4.83 (t, J = 6.8 Hz, 1 H), 4.49 (m, 1 H), 4.03 (m, 2 | |
| | | | yl]methyl morpholine-4-carboxylate | | H), 3.91 (m, 1 H), 3.34-3.60 (m, 4 H), | |
| | | | trifluoroacetate | | 3.17 (m, 4 H), 2.74 (m, 4 H), 2.55 (m, 3 H), | |
| | | | | | 2.21-2.46 (m, 4 H), 1.52-1.99 (m, 7 H) | |
| 12 | pure | 4R | (4R)-4-[(4,4-difluoropiperidin-1- | 450 | 7.28 (m, 2 H), 6.79 (m, 2 H), 4.73 (m, 1 H), | −26.10 |
| | | trans | yl)methyl]-3-{4-[(trans-3-piperidin-1- | | 4.52 (m, 1 H), 4.38 (m, 1 H), 4.29 (m, 1 H), | |
| | | | ylcyclobutyl)oxy]phenyl}-1,3- | | 2.98 (m, 1 H), 2.65 (m, 1 H), 2.57 (m, 3 H), | |
| | | | oxazolidin-2-one | | 2.48 (m, 2 H), 2.39 (m, 3 H), 2.29 (m, 5 H), | |
| | | | | | 1.92 (m, 4 H), 1.60 (m, 4 H), 1.46 (m, 2 H) | |
| 13 | pure | 4R | (4R)-4-(morpholin-4-ylmethyl)-3-{4- | 416 | (CDCl3) 7.29 (d, J = 8.8 Hz, 2 H), 6.79 (d, J = 9.0 Hz, | −31.20 |
| | | trans | [(trans-3-piperidin-1- | | 2 H), 4.73 (m, 1 H), 4.52 (m, 1 H), | |
| | | | ylcyclobutyl)oxy]phenyl}-1,3- | | 4.37 (m, 2 H), 3.63 (m, 4 H), 2.98 (m, 1 H), | |
| | | | oxazolidin-2-one | | 2.61 (m, 1 H), 2.48 (m, 3 H), 2.38 (m, 5 H), | |
| | | | | | 2.29 (m, 5 H), 1.62 (m, 4 H), 1.49 (m, 2 H) | |
| 14 | pure | 4R | (4R)-4-(cyclohexylmethyl)-3-{4- | 413 | 7.28 (m, 2 H), 6.84 (m, 2 H), 4.71 (m, 1 H), | −27.57 |
| | | trans | [(trans-3-piperidin-1- | | 4.53 (m, 2 H), 4.07 (m, 1 H), 2.86 (m, 1 H), | |
| | | | ylcyclobutyl)oxy]phenyl}-1,3- | | 2.34 (m, 2 H), 2.22 (m, 4 H), 2.11 (m, 2 H), | |
| | | | oxazolidin-2-one | | 1.73 (m, 1 H), 1.57 (d, J = 11.8 Hz, 4 H), | |
| | | | | | 1.49 (m, 4 H), 1.37 (m, 4 H), 1.23 (m, 1 H), | |
| | | | | | 1.09 (m, 3 H), 0.82 (m, 2 H) | |
| 15 | pure | 4R | (4R)-3-{4-[(trans-3-piperidin-1- | 414 | 7.30 (d, J = 9.0 Hz, 2 H), 6.78 (m, 2 H), | −31.96 |
| | | trans | ylcyclobutyl)oxy]phenyl}-4-(piperidin- | | 4.73 (m, 1 H), 4.52 (m, 1 H), 4.35 (m, 2 H), | |
| | | | 1-ylmethyl)-1,3-oxazolidin-2-one | | 3.00 (m, 1 H), 2.54 (m, 1 H), 2.39 (m, 6 H), | |
| | | | | | 2.29 (m, 7 H), 1.61 (m, 4 H), 1.51 (m, 6 H), | |
| | | | | | 1.36 (m, 2 H) | |
| 16 | pure | 5S | (5S)-5-(morpholin-4-ylmethyl)-1-{4- | 430 | 7.35 (m, 2 H), 7.22 (d, J = 8.8 Hz, 2 H), | +0.10 |
| | | trans | [(trans-3-piperidin-1- | | 4.31 (m, 1 H), 3.82 (m, 1 H), 3.58 (t, J = 4.5 Hz, | |
| | | | ylcyclobutyl)thio]phenyl}pyrrolidin-2- | | 4 H), 3.07 (quint, J = 7.5 Hz, 1 H), | |
| | | | one | | 2.69 (m, 1 H), 2.02-2.55 (m, 17 H), 1.60 (m, 4 H), | |
| | | | | | 1.45 (m, 2 H) | |
| 17 | pure | 5S | (5S)-5-[(4,4-difluoropiperidin-1- | 464 | 7.37 (d, J = 8.6 Hz, 2 H), 7.21 (d, J = 8.6 Hz, | +14.91 |
| | | trans | yl)methyl]-1-{4-[(trans-3-piperidin-1- | | 2 H), 4.31 (m, 1 H), 3.92 (m, 1 H), 3.57 (m, 1 | |
| | | | ylcyclobutyl)thio]phenyl}pyrrolidin-2- | | H), 3.13 (m, 2 H), 2.67 (m, 1 H), | |
| | | | one | | 2.16-2.58 (m, 11 H), 2.02 (m, 1 H), 1.78-1.94 (m, 9 H), | |
| | | | | | 1.48-1.74 (m, 4 H) | |
| 18 | pure | 4S | (4S)-4-(morpholin-4-ylcarbonyl)-3-{4- | 430 | 7.07 (m, 2 H), 6.58 (m, 2 H), 5.32 (m, 1 H), | |
| | | trans | [(trans-3-piperidin-1- | | 4.47 (m, 1 H), 4.35 (m, 1 H), 4.09 (m, 1 H), | |
| | | | ylcyclobutyl)oxy]phenyl}-1,3- | | 3.37 (m, 2 H), 3.22 (m, 6 H), 2.61 (m, 1 H), | |
| | | | oxazolidin-2-one | | 2.09 (m, 2 H), 1.95 (m, 4 H), 1.86 (m, 2 H), | |
| | | | | | 1.26 (m, 4 H), 1.12 (m, 2 H) | |
| 19 | pure | 4S | (4S)-4-(piperidin-1-ylcarbonyl)-3-{4- | 428 | 7.06 (m, 2 H), 6.57 (m, 2 H), 5.33 (m, 1 H), | |
| | | trans | [(trans-3-piperidin-1- | | 4.46 (m, 1 H), 4.33 (m, 1 H), 4.00 (m, 1 H), | |
| | | | ylcyclobutyl)oxy]phenyl}-1,3- | | 3.20 (m, 4 H), 2.62 (m, 1 H), 2.09 (m, 2 H), | |
| | | | oxazolidin-2-one | | 1.94 (m, 4 H), 1.86 (m, 2 H), 1.34 (m, 2 H), | |
| | | | | | 1.24 (m, 6 H), 1.12 (m, 4 H) | |
| 20 | pure | 4S | (4S)-4-[(4,4-difluoropiperidin-1- | 464 | 7.32 (m, 2 H), 6.81 (m, 2 H), 5.60 (m, 1 H), | |
| | | trans | yl)carbonyl]-3-{4-[(trans-3-piperidin- | | 4.70 (m, 1 H), 4.57 (m, 1 H), 4.38 (m, 1 H), | |
| | | | 1-ylcyclobutyl)oxy]phenyl}-1,3- | | 3.55 (m, 4 H), 2.83 (m, 1 H), 2.32 (m, 2 H), | |
| | | | oxazolidin-2-one | | 2.21 (m, 4 H), 2.09 (m, 2 H), 1.90 (m, 4 H), | |
| | | | | | 1.49 (m, 4 H), 1.36 (m, 2 H) | |
| 21 | mixture | 5S,2, | (5S)-5-[(4,4-difluoropiperidin-1- | | 7.24 (d, J = 9.0 Hz, 2 H), 6.78 (d, J = 8.8 Hz, | |
| | | trans | yl)methyl]-1-(4-{[trans-3-(2- | | 2 H), 4.73 (m, 1 H), 4.21 (m, 1 H), 3.39 (m, 1 | |
| | | | methylpyrrolidin-1- | | H), 3.00 (m, 1 H), 2.64 (m, 1 H), 2.46 (m, 10 | |
| | | | yl)cyclobutyl]oxy}phenyl)pyrrolidin-2- | | H), 2.29 (m, 3 H), 1.90 (m, 7 H), 1.69 (m, 2 | |
| | | | one | | H), 1.45 (m, 1 H), 1.08 (d, J = 6.2 Hz, 3 H) | |
| 22 | pure | A- | (−)-(5S)-5-[(4,4-difluoropiperidin-1- | | 7.24 (d, J = 9.0 Hz, 2 H), 6.78 (d, J = 8.8 Hz, | |
| | | 5S,2!, | yl)methyl]-1-(4-{[trans-3-(2- | | 2 H), 4.73 (m, 1 H), 4.21 (m, 1 H), 3.39 (m, 1 | |
| | | trans | methylpyrrolidin-1- | | H), 3.00 (m, 1 H), 2.64 (m, 1 H), 2.46 (m, 10 | |
| | | | yl)cyclobutyl]oxy}phenyl)pyrrolidin-2- | | H), 2.29 (m, 3 H), 1.90 (m, 7 H), 1.69 (m, 2 | |
| | | | one | | H), 1.45 (m, 1 H), 1.08 (d, J = 6.2 Hz, 3 H) | |
| 23 | pure | B- | (+)-(5S)-5-[(4,4-difluoropiperidin-1- | | 7.24 (d, J = 9.0 Hz, 2 H), 6.78 (d, J = 8.8 Hz, | |
| | | 5S,2!, | yl)methyl]-1-(4-{[trans-3-(2- | | 2 H), 4.73 (m, 1 H), 4.21 (m, 1 H), 3.39 (m, 1 | |
| | | trans | methylpyrrolidin-1- | | H), 3.00 (m, 1 H), 2.64 (m, 1 H), 2.46 (m, 10 | |
| | | | yl)cyclobutyl]oxy}phenyl)pyrrolidin-2- | | H), 2.29 (m, 3 H), 1.90 (m, 7 H), 1.69 (m, 2 | |
| | | | one | | H), 1.45 (m, 1 H), 1.08 (d, J = 6.2 Hz, 3 H) | |

### Example 7

### Affinity for the Histamine H₃-receptor; Inverse agonism, antagonism and agonism activity: [³⁵S]GTPγS-binding assay human Histamine H₃-receptor.

### Material and Methods

### Reagents

Reagents and reference compounds are of analytical grade and may be obtained from various commercial sources. [³H]-N-α-methylhistamine (80-85 Ci/mmol) and [³⁵S]-GTPγS (1250 Ci/mmol) are purchased from Perkin Elmer (Belgium). Cell culture reagents are purchased from Cambrex (Belgium).

Test and reference compounds are dissolved in 100% DMSO to give a 1 mM stock solution. Final DMSO concentration in the assay does not exceed 1%. A CHO cell line expressing the unspliced full length (445 AA) human H₃ histamine receptor may be obtained e.g. from Euroscreen S.A. (Belgium).

### Cell Culture

Cells are grown in HAM-F12 culture media containing 10% fetal bovine serum, 100 IU/ml penicillin, 100 μg/ml streptomycin, 1% sodium pyruvate and 400 μg/ml of gentamycin. Cells are maintained at 37° C. in a humidified atmosphere composed of 95% air and 5% CO₂.

### Membrane Preparation

Confluent cells are detached by 10 min incubation at 37° C. in PBS/EDTA 0.02%. The cell suspension is centrifuged at 1,500×g for 10 min at 4° C. The pellet is homogenized in a 15 mM Tris-HCl buffer (pH 7.5) containing 2 mM MgCl₂, 0.3 mM EDTA, 1 mM EGTA (buffer A). The crude homogenate is frozen in liquid nitrogen and thawed. DNAse (1 μl/ml) is then added and the homogenate is further incubated for 10 min at 25° C. before being centrifuged at 40,000×g for 25 min at 4° C. The pellet is resuspended in buffer A and washed once more under the same conditions. The final membrane pellet is resuspended, at a protein concentration of 1-3 mg/ml, in a 7.5 mM Tris-HCl buffer (pH 7.5) enriched with 12.5 mM MgCl₂, 0.3 mM EDTA, 1 mM EGTA and 250 mM sucrose and stored in liquid nitrogen until used.

### Binding Assays

[³H]-N-α-methylhistamine binding assay

Affinity of compounds for human histamine H₃ receptors may be measured by competition with [³H]-N-α-methylhistamine. This binding assay may be performed on any H3 sequence, human or non-human. Briefly, membranes (20-40 μg proteins) expressing human H₃ histamine receptors are incubated at 25° C. in 0.5 ml of a 50 mM Tris-HCl buffer (pH 7.4) containing 2 mM MgCl₂, 0.2 nM [³H]-N-α-methyl-histamine and increasing concentrations of drugs. The non specific binding (NSB) is defined as the residual binding observed in the presence of 10 μM thioperamide or histamine. Membrane-bound and free radioligand are separated by rapid filtration through glass fiber filters presoaked in 0.1% PEI. Samples and filters are rinsed by at least 6 ml of ice-cold 50 mM Tris-HCl buffer (pH 7.4). The entire filtration procedure does not exceed 10 seconds per sample. Radioactivity trapped onto the filters is counted by liquid scintillation in a R-counter.

[³⁵S]-GTPγS binding assay

Stimulation (agonist) or inhibition (inverse agonist) of [³⁵S]-GTPγS binding to membrane expressing human H₃ histamine receptors is measured as described by Lorenzen et al. (Mol. Pharmacol. 1993, 44, 115-123) with a few modifications. Briefly, membranes (10-20 μg proteins) expressing human H₃ histamine receptors are incubated at 25° C. in 0.2 ml of a 50 mM Tris-HCl buffer (pH 7.4) containing 3 mM MgCl₂, 50 mM NaCl, 1 μM GDP, 2 μg saponin and increasing concentrations of drugs. After 15 min pre-incubation, 0.2 nM of [³⁵S]-GTPγS are added to the samples. The non specific binding (NSB) is defined as the residual binding observed in the presence of 100 μM Gpp(NH)p. Membrane-bound and free radioligand are separated by rapid filtration through glass fiber filters. Samples and filters are rinsed by at least 6 ml of ice-cold 50 mM Tris-HCl buffer (pH 7.4). The entire filtration procedure does not exceed 10 seconds per sample. Radioactivity trapped onto the filters is counted by liquid scintillation in a β-counter.

Data analysis
Determination of plC₅₀/pKi/pEC₅₀/pEC₅₀INV

### Analysis

Raw data are analyzed by non-linear regression using XLfit™ (IDBS, United Kingdom) according to the following generic equation

B=MIN+[(MAX−MIN)/(1+(((10^{x})/(10^{-pX50}))^{nH}))]

where:
B is the radioligand bound in the presence of the unlabelled compound (dpm),
MIN is the minimal binding observed (dpm)
MAX is maximal binding observed (dpm),
X is the concentration of unlabelled compound (log M),
pX₅₀ (−log M) is the concentration of unlabelled compound causing 50% of its maximal effect (inhibition or stimulation of radioligand binding). It stands for plC₅₀ when determining the affinity of a compound for the receptor in binding studies with [³H]-N-α-methylhistamine, for pEC₅₀ for compounds stimulating the binding of [³⁵S]-GTPγS (agonists) and for pEC₅₀INV for compounds inhibiting the binding of [³⁵S]-GTPγS (inverse agonists).
n_{H} is the Hill coefficient.

pKi may be obtained by applying the following equation (Cheng and Prusoff, 1973, Biochem. Pharmacol., 22: 3099-3108):

pKi=plC₅₀+log(1+L/Kd)

where:
pKi is the unlabelled compound equilibrium dissociation constant (−log M),
L is the radioligand concentration (nM),
Kd is the radioligand equilibrium dissociation constant (nM).

Compounds of formula (I) according to the invention show plC₅₀ values of at least 6.5, more preferably of at least 8 or 9, typically greater than 7.5 for the histamine H3 receptor.

Compounds of formula (I) according to the invention showed pEC₅₀INV values typically greater than 7.5 for the histamine H₃ receptor.

### Example 8

### Antagonism activity: Paced isolated guinea pig myenteric plexus—Electric-Field Stimulation assay

### Material and methods

### Reagents

Stock solutions (10⁻² M) of compounds to be tested and further dilutions are freshly prepared in DMSO (WNR, Leuven, Belgium). All other reagents (R(−)-α-methylhistamine, mepyramine, ranitidine, propranolol, yohimbine and components of the Krebs' solution) are of analytical grade and obtained from conventional commercial sources.

### Animals

Four week-old male Dunkin-Hartley guinea pigs (200-300 g) are supplied by Charles River (Sultfeld, Germany). All animals are ordered and used under protocol “orgisol-GP” approved by the UCB Pharma ethical committee. Animals are housed in the UCB animal facility in groups of 12, in stainless steel cages (75×50×30 cm) and allowed to acclimatise for a minimum of one week before inclusion in the study. Room temperature is maintained between 20 and 24° C. with 40 to 70% relative humidity. A light and dark cycle of 12 h is applied. Animals have free access to food and water.

### Organ Preparation

The method is adapted from that described by Menkveld et al. in Eur. J. Pharmacol. 1990, 186, 343-347. Longitudinal myenteric plexus is prepared from the isolated guinea pig ileum. Tissues are mounted in 20-ml organ baths containing modified Krebs' solution with 10⁻⁷ M mepyramine, 10⁻⁵ M ranitidine, 10⁻⁵ M propranolol and 10⁻⁶ M yohimbine. The bathing solution is maintained at 37° C. and gassed with 95% O₂-5% CO₂. Tissues are allowed to equilibrate for a 60-min period under a resting tension of 0.5 g and an electrical field stimulation (pulses of 5-20 V, 1 ms and 0.1 Hz is applied during the whole experiment). Such a stimulation induces stable and reproductive twitch contractions. Isometric contractions are measured by force-displacement transducers coupled to an amplifier connected to a computer system (EMKA Technologies) capable of controlling (i) automatic data acquisition, (ii) bath washout by automatic fluid circulation through electrovalves at predetermined times or signal stability and (iii) automatic dilution/injection of drug in the bath at predetermined times or signal stability.

### Protocol

After a 60 min-stabilisation period, tissues are stimulated twice with 10⁻⁶ M R(−)-α-methylhistamine at 30-min interval. After a 60-min incubation period in the presence of solvent or antagonist test compound, a cumulative concentration-response to R(−)-α-methylhistamine is elicited (10⁻¹⁰ à 10⁻⁴ M). Only one concentration of antagonist is tested on each tissue.

### Data Analysis

An appropriate estimate of interactions between agonist and antagonist can be made by studying the family of curves observed in the absence or presence of increasing antagonist concentrations. The value of each relevant parameter of each concentration-response curve (pD₂ and Eₘₐₓ) is calculated by an iterative computer software (XLfit, IDBS, Guildford, UK) fitting the experimental data to the four parameter logistic equation. Antagonistic activity of the test substance is estimated by the calculation of pD′₂ and/or pA₂ values according to the methods described by Van Rossum et al. in Arch. Int. Pharmacodyn. Ther. 1963, 143, 299 and/or by Arunlakshana & Schild in Br. J. Pharmacol 1959, 14, 48

Results are expressed as the mean±SD. The number of observations is indicated as n.

Compounds of formula (I) according to the invention showed pA₂ values typically greater than or equal to 7.5 for the histamine H₃ receptor.

### Example 9

### hERG Study

This is an in vitro electrophysiological patch clamp study to assess the potential effects of test compounds on human ether-a-go-go-related gene (hERG)-encoded channel tail current recorded from HEK293 cells stably transfected with hERG cDNA. Coverslips on which cells are seeded are mounted in a recording chamber and superfused with physiogical saline. Recordings of tail current are made in the voltage patch clamp mode. A reference substance e.g. E-4031 is used to confirm that the current observed can be inhibited by a known hERG channel blocker (Zhou, Z. et al., Biophys. J., 1998, 74, 230-241).

Compounds of the current invention typically show weak hERG channel affinities (generally greater than or equal to 1 μM).

1. A compound of formula (I′), geometrical isomers, enantiomers, diastereoisomers, pharmaceutically acceptable salts and all possible mixtures thereof, 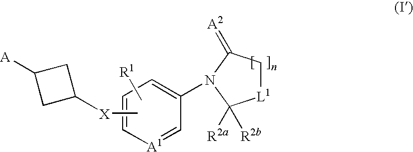
wherein
A¹ is CH, C-halogen or N;
A² is oxygen or sulfur;
X is O, S, NH or N(C₁₋₄ alkyl);
R¹ is hydrogen, halogen, C₁₋₄ alkyl or C₁₋₄ alkoxy;
R^{2a} is hydrogen, C₁₋₆ alkyl, C₁₋₆-alkyl cycloalkyl, aryl, C₂₋₆ alkenyl, heteroaryl, C₃₋₈ cycloalkyl, 3-8-membered heterocycloalkyl, acyl, C₁₋₆-alkyl aryl, C₁₋₆-alkyl heteroaryl, C₁₋₆-alkyl heterocycloalkyl, alkoxycarbonyl, aminocarbonyl, C₁₋₆-alkyl acyl, C₁₋₆-alkyl alkoxy, C₁₋₆-alkyl alkoxycarbonyl, C₁₋₆-alkyl aminocarbonyl, C₁₋₆-alkyl acylamino, C₁₋₆-alkyl ureido, C₁₋₆-alkyl aminocarbonyloxy, C₁₋₆-alkyl aminocarbonylthio, C₁₋₆-alkyl carbamate, C₁₋₆-alkyl amino, C₃₋₈-cycloalkyl amino, C₁₋₆-alkyl hydroxy or cyano;
R^{2b} is hydrogen, C₁₋₈ alkyl or C₃₋₈ cycloalkyl;
or R^{2a} and R^{2b} are linked together to form a C₃₋₈ cycloalkyl or a 3-8 membered heterocycloalkyl;
A is a substituted or unsubstituted aliphatic or cyclic amino group which is linked to the cyclobutyl group via an amino nitrogen;
L¹ is —(O)ᵥ—(CR^{9a}R^{9b})ₘ—(CH₂)_{z};
R^{9a} is hydrogen or C₁₋₈ alkyl;
R^{9b} is a C₁₋₆-alkyl aryl or C₁₋₈ alkyl;
n is an integer equal to 0 or 1;
v is an integer equal to 0 or 1;
m is an integer equal to 0 or 1;
z is an integer equal to 0, 1, 2 or 3.

2. The compound of formula (I) according to claim 1, 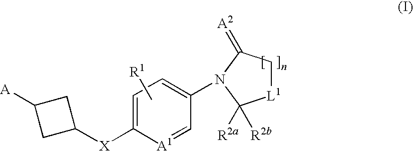
wherein
A¹ is CH, C-halogen or N;
A² is oxygen or sulfur;
X is O, S, NH or N(C₁₋₄ alkyl);
R¹ is hydrogen, halogen, C₁₋₄ alkyl or C₁₋₄ alkoxy;
R^{2a} is hydrogen, C₁₋₆ alkyl, C₁₋₆-alkyl cycloalkyl, aryl, C₂₋₆ alkenyl, heteroaryl, C₃₋₈ cycloalkyl, 3-8-membered heterocycloalkyl, acyl, C₁₋₆-alkyl aryl, C₁₋₆-alkyl heteroaryl, C₁₋₆-alkyl heterocycloalkyl, alkoxycarbonyl, aminocarbonyl, C₁₋₆-alkyl acyl, C₁₋₆-alkyl alkoxy, C₁₋₆-alkyl alkoxycarbonyl, C₁₋₆-alkyl aminocarbonyl, C₁₋₆-alkyl acylamino, C₁₋₆-alkyl ureido, C₁₋₆-alkyl aminocarbonyloxy, C₁₋₆-alkyl aminocarbonylthio, C₁₋₆-alkyl carbamate, C₁₋₆-alkyl amino, C₃₋₈-cycloalkyl amino, C₁₋₆-alkyl hydroxy or cyano;
R^{2b} is hydrogen, C₁₋₈ alkyl or C₃₋₈ cycloalkyl;
or R^{2a} and R^{2b} are linked together to form a C₃₋₈ cycloalkyl or a 3-8 membered heterocycloalkyl;
A is a substituted or unsubstituted aliphatic or cyclic amino group which is linked to the cyclobutyl group via an amino nitrogen;
L¹ is —(O)ᵥ—(CR^{9a}R^{9b})ₘ—(CH₂)_{z};
R^{9a} is hydrogen or C₁₋₈ alkyl;
R^{9b} is a C₁₋₆-alkyl aryl or C₁₋₈ alkyl;
n is an integer equal to 0 or 1;
v is an integer equal to 0 or 1;
m is an integer equal to 0 or 1;
z is an integer equal to 0, 1, 2 or 3.

3. The compound according to claim 1, wherein A¹ is CH, C—F or N.

4. The compound according to claim 1, wherein A² is oxygen.

5. The compound according to claim 1, wherein X is O or S.

6. The compound according to claim 1, wherein R¹ is hydrogen or halogen.

7. The compound according to claim 1, wherein R^{2b} is hydrogen.

8. The compound according to claim 1, wherein R^{2a} is hydrogen, C₁₋₆ alkyl, C₁₋₆-alkyl cycloalkyl, C₁₋₆-alkyl heterocycloalkyl, C₁₋₆-alkyl amino, aminocarbonyl, C₁₋₆-alkyl ureido or C₁₋₆-alkyl aminocarbonyloxy.

9. The compound according to claim 1, wherein A is a 3 to 8 membered heterocycloalkyl linked to the cyclobutyl group via a nitrogen atom.

10. The compound according to claim 9, wherein A is a 3 to 8 membered heterocycloalkyl selected from piperidin-1-yl, morpholin-4-yl, pyrrolidin-1-yl and piperazin-1-yl.

11. The compound according to claim 10, wherein A is piperidin-1-yl, 2-methylpyrrolidin-1-yl, (2R)-2-methylpyrrolidin-1-yl and (2S)-2-methylpyrrolidin-1-yl.

12. The compound of formula (Ib), according to claim 1: 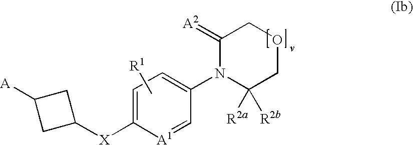
wherein A¹, A², X, R¹, R^{2a}, R^{2b}, A and v are as defined in claim 1.

13. The compound according to claim 1 selected from the group consisting of:
(5S)-1-{4-[(trans-3-piperidin-1-ylcyclobutyl)oxy]phenyl}-5-(piperidin-1-ylmethyl)pyrrolidin-2-one;
(5S)-5-(cyclohexylmethyl)-4-{4-[(trans-3-piperidin-1-ylcyclobutyl)oxy]-phenyl}morpholin-3-one;
(5S)-5-[(4,4-difluoropiperidin-1-yl)methyl]-1-{4-[(trans-3-piperidin-1-ylcyclobutyl)oxy]-phenyl}pyrrolidin-2-one;
(5S)-5-(morpholin-4-ylmethyl)-1-{4-[(trans-3-piperidin-1-ylcyclobutyl)oxy]-phenyl}pyrrolidin-2-one;
(5S)-1-{4-[(trans-3-piperidin-1-ylcyclobutyl)oxy]phenyl}-5-(pyrrolidin-1-ylmethyl)pyrrolidin-2-one;
(5S)-1-{4-[(trans-3-piperidin-1-ylcyclobutyl)oxy]phenyl}-5-{[(2,2,2-trifluoroethyl)-amino]methyl}-pyrrolidin-2-one;
(5S)-5-[(3,3-difluoropyrrolidin-1-yl)methyl]-1-{4-[(trans-3-piperidin-1-ylcyclobutyl)oxy]phenyl}-pyrrolidin-2-one;
(5S)-1-{3-[(trans-3-piperidin-1-ylcyclobutyl)oxy]phenyl}-5-(piperidin-1-ylmethyl)pyrrolidin-2-one;
5-[(4,4-difluoropiperidin-1-yl)methyl]-4-{4-[(trans-3-piperidin-1-ylcyclobutyl)-oxy]phenyl}morpholin-3-one;
(5R)-5-(cyclohexylmethyl)-4-{4-[(trans-3-piperidin-1-ylcyclobutyl)oxy]phenyl}-morpholin-3-one;
[(2S)-5-oxo-1-{4-[(trans-3-piperidin-1-ylcyclobutyl)oxy]phenyl}pyrrolidin-2-yl]methyl morpholine-4-carboxylate;
(4R)-4-[(4,4-difluoropiperidin-1-yl)methyl]-3-{4-[(trans-3-piperidin-1-ylcyclobutyl)-oxy]phenyl}-1,3-oxazolidin-2-one;
(4R)-4-(morpholin-4-ylmethyl)-3-{4-[(trans-3-piperidin-1-ylcyclobutyl)oxy]phenyl}-1,3-oxazolidin-2-one;
(4R)-4-(cyclohexylmethyl)-3-{4-[(trans-3-piperidin-1-ylcyclobutyl)oxy]phenyl}-1,3-oxazolidin-2-one;
(4R)-3-{4-[(trans-3-piperidin-1-ylcyclobutyl)oxy]phenyl}-4-(piperidin-1-ylmethyl)-1,3-oxazolidin-2-one;
(5S)-5-(morpholin-4-ylmethyl)-1-{4-[(trans-3-piperidin-1-ylcyclobutyl)thio]phenyl}-pyrrolidin-2-one;
(5S)-5-[(4,4-difluoropiperidin-1-yl)methyl]-1-{4-[(trans-3-piperidin-1-ylcyclobutyl)-thio]phenyl}pyrrolidin-2-one;
(4S)-4-(morpholin-4-ylcarbonyl)-3-{4-[(trans-3-piperidin-1-ylcyclobutyl)oxy]-phenyl}-1,3-oxazolidin-2-one;
(4S)-4-(piperidin-1-ylcarbonyl)-3-{4-[(trans-3-piperidin-1-ylcyclobutyl)oxy]phenyl}-1,3-oxazolidin-2-one;
(4S)-4-[(4,4-difluoropiperidin-1-yl)carbonyl]-3-{4-[(trans-3-piperidin-1-ylcyclobutyl)-oxy]phenyl}-1,3-oxazolidin-2-one;
(5S)-5-[(4,4-difluoropiperidin-1-yl)methyl]-1-(4-{[trans-3-(2-methylpyrrolidin-1-yl)cyclobutyl]oxy}phenyl)pyrrolidin-2-one;
(5S)-5-[(4,4-difluoropiperidin-1-yl)methyl]-1-(4-{[trans-3-((2R)-2-methylpyrrolidin-1-yl)cyclobutyl]oxy}phenyl)pyrrolidin-2-one; and
(5S)-5-[(4,4-difluoropiperidin-1-yl)methyl]-1-(4-{[trans-3-((2S)-2-methylpyrrolidin-1-yl)cyclobutyl]oxy}phenyl)pyrrolidin-2-one.

14. A pharmaceutical composition comprising an effective amount of a compound according to claim 1 or a pharmaceutically acceptable salt thereof in combination with a pharmaceutically acceptable diluent or carrier.

15. The compound according to any of claim 1 for use as a medicament.

16. The compound according to claim 1 for the treatment and prevention of mild-cognitive impairment, Alzheimer's disease, learning and memory disorders, attention-deficit hyperactivity disorder, Parkinson's disease, schizophrenia, dementia, depression, epilepsy, seizure disorders, convulsions, sleep/wake disorders, cognitive dysfunctions, narcolepsy, hypersomnia, obesity, upper airway allergic disorders, Down's syndrome, anxiety, stress, cardiovascular disorders, inflammation and pain.

17. A synthetic intermediate of formula (II) geometrical isomers, enantiomers, diastereoisomers, pharmaceutically acceptable salts and all possible mixtures thereof, 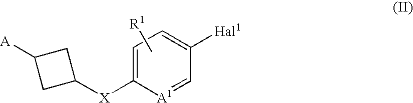
wherein A, A¹, X, and R¹ are as defined in claim 1 and Hal¹ is a halogen.

18. The synthetic intermediate according to claim 17 selected from the list consisting of:
3-(2-methylpyrrolidin-1-yl)cyclobut-2-en-1-one;
3-morpholin-4-ylcyclobut-2-en-1-one;
3-(4-isopropylpiperazin-1-yl)cyclobut-2-en-1-one;
3-(4,4-difluoropiperidin-1-yl)cyclobut-2-en-1-one;
3-azepan-1-ylcyclobut-2-en-1-one;
3-[(3R)-3-(dimethylamino)pyrrolidin-1-yl]cyclobut-2-en-1-one;
3-thiomorpholin-4-ylcyclobut-2-en-1-one;
cis-3-piperidin-1-ylcyclobutanol;
cis-3-(2-methylpyrrolidin-1-yl)cyclobutanol;
cis-3-morpholin-4-ylcyclobutanol;
cis-3-(4-isopropylpiperazin-1-yl)cyclobutanol;
cis-3-(4,4-difluoropiperidin-1-yl)cyclobutanol;
cis-3-pyrrolidin-1-ylcyclobutanol;
cis-3-azepan-1-ylcyclobutanol;
cis-3-[(3R)-3-(dimethylamino)pyrrolidin-1-yl]cyclobutanol;
cis-3-thiomorpholin-4-ylcyclobutanol;
cis-3-piperidin-1-ylcyclobutyl 4-methylbenzenesulfonate;
cis-3-(2-methylpyrrolidin-1-yl)cyclobutyl 4-methylbenzenesulfonate;
cis-3-(2-methylpyrrolidin-1-yl)cyclobutyl 4-bromobenzenesulfonate;
cis-3-morpholin-4-ylcyclobutyl 4-methylbenzenesulfonate;
cis-3-(4-isopropylpiperazin-1-yl)cyclobutyl 4-methylbenzenesulfonate;
cis-3-(4,4-difluoropiperidin-1-yl)cyclobutyl 4-methylbenzenesulfonate;
cis-3-pyrrolidin-1-ylcyclobutyl 4-methylbenzenesulfonate;
cis-3-azepan-1-ylcyclobutyl 4-methylbenzenesulfonate;
cis-3-[(3R)-3-(dimethylamino)pyrrolidin-1-yl]cyclobutyl 4-methylbenzenesulfonate;
cis-3-thiomorpholin-4-ylcyclobutyl 4-methylbenzenesulfonate;
1-[trans-3-(4-bromophenoxy)cyclobutyl]piperidine;
1-[trans-3-(3-bromophenoxy)cyclobutyl]piperidine;
1-[trans-3-(4-iodophenoxy)cyclobutyl]piperidine;
1-[trans-3-(4-iodophenoxy)cyclobutyl]-2-methylpyrrolidine;
(2R)-1-[trans-3-(4-iodophenoxy)cyclobutyl]-2-methylpyrrolidine;
(2S)-1-[trans-3-(4-iodophenoxy)cyclobutyl]-2-methylpyrrolidine;
(5S)-5-[(3,3-difluoropyrrolidin-1-yl)methyl]pyrrolidin-2-one;
2-chloro-N-[(1S)-2-cyclohexyl-1-(hydroxymethyl)ethyl]acetamide;
(5S)-5-(cyclohexylmethyl)morpholin-3-one;
(5R)-5-(cyclohexylmethyl)morpholin-3-one;
[(2S)-5-oxopyrrolidin-2-yl]methyl morpholine-4-carboxylate;
(4R)-4-(morpholin-4-ylmethyl)-1,3-oxazolidin-2-one;
propan-2-yl 5-oxomorpholine-3-carboxylate;
5-(hydroxymethyl)morpholin-3-one;
(5-oxomorpholin-3-yl)methyl 4-methylbenzenesulfonate;
5-[(4,4-difluoropiperidin-1-yl)methyl]morpholin-3-one;
1-[trans-3-(tritylsulfanyl)cyclobutyl]piperidine;
trans-3-[butyl(ethyl)amino]cyclobutanethiol;
1-{trans-3-[(4-iodophenyl)sulfanyl]cyclobutyl}piperidine;
tert-butyl (4S)-2,2-dimethyl-4-(morpholin-4-ylcarbonyl)-1,3-oxazolidine-3-carboxylate;
tert-butyl (4S)-2,2-dimethyl-4-(piperidin-1-ylcarbonyl)-1,3-oxazolidine-3-carboxylate;
tert-butyl (4S)-4-[(4,4-difluoropiperidin-1-yl)carbonyl]-2,2-dimethyl-1,3-oxazolidine-3-carboxylate;
(2S)-2-amino-1-(4,4-difluoropiperidin-1-yl)-3-hydroxypropan-1-one hydrochloride;
(4S)-4-(morpholin-4-ylcarbonyl)-1,3-oxazolidin-2-one;
(4S)-4-(piperidin-1-ylcarbonyl)-1,3-oxazolidin-2-one; and
(4S)-4-[(4,4-difluoropiperidin-1-yl)carbonyl]-1,3-oxazolidin-2-one.
